SEQUENCE LISTING (2 sequences)

SEQ ID NO: 1 - DNA, 20 bp, Artificial Sequence
  Description of Artificial Sequence primer.
tcattcgagtagcggctctt

SEQ ID NO: 2 - DNA, 20 bp, Artificial Sequence
  Description of Artificial Sequence primer.
cttctttgctcctccattgc

(19) United States
(12) Patent Application Publication
(10) Pub. No.: US 2012/0064535 A1
(43) Pub. Date: Mar. 15, 2012

(54) METHOD OF PREPARING SAMPLES CONTAINING NUCLEIC ACIDS

(75) Inventors: Yasuo Tanigami, Tokyo (JP); Tomonori Nagaoka, Tokyo (JP)

(73) Assignee: OLYMPUS CORPORATION, Tokyo (JP)

(21) Appl. No.: 13/300,006

(22) Filed: Nov. 18, 2011

Related U.S. Application Data
(63) Continuation of application No. PCT/JP2010/001754, filed on Mar. 11, 2010.

(30) Foreign Application Priority Data
May 20, 2009 (JP) 2009-122438

(51) Int. Cl. C12Q 1/68; C07H 21/00

(52) U.S. Cl. 435/6.12; 536/25.4; 536/23.1

(57) ABSTRACT

The present invention provides a method of preparing a sample enabling efficient recovery of nucleic acids from a biological sample such as stool without requiring a bothersome procedure. The method of preparing a nucleic acid-containing sample of the present invention comprises (A) a step for mixing a biological sample with a nucleic acid stabilizer, (B) a step for recovering a solid component from the mixture obtained in step (A) in the form of a nucleic acid-containing sample, and (C) a step for washing the solid component recovered in step (B) using an acidic buffer solution having a pH of 2 or higher.

### BACKGROUND OF THE INVENTION

1. Field of the Invention

The present invention relates to a method of preparing nucleic acid-containing samples from a biological sample in order to efficiently recover the nucleic acids contained in the biological sample, a nucleic acid-containing sample prepared according to this preparation method, and a method of recovering nucleic acids from nucleic acid-containing samples prepared using this preparation method.

The present application claims priority on the basis of Japanese Patent Application No. 2009-122438, filed in Japan on May 20, 2009, the contents of which are incorporated herein by reference.

2. Description of the Related Art

Due to recent progress made in the field of genetic analysis technology, attempts are being made to analyze nucleic acids in biological samples to be of use in the diagnosis and treatment of diseases. The use of nucleic acids in stool, blood or other biological samples offers the advantages of being less invasive and placing less of a burden on patients in comparison with endoscopic examinations and other clinical tests. In addition, since nucleic acid analysis enables direct examination of genes relating to diseases, they offer the advantage of allowing the obtaining of highly reliable results. For example, investigating for the presence of cancer cells or cancer cell-derived genes in stool and blood samples enables early detection of cancer and determination of the degree of its progression.

On the other hand, in order to accurately detect cancer cells and the like in stool and other biological samples, it is important to efficiently recover cancer cell-derived nucleic acids present in those biological samples. In particular, since cancer cell-derived nucleic acids are only present in minute amounts, if the efficiency at which nucleic acids are recovered from a biological sample is poor, cancer cell-derived nucleic acids end up being undetected even though they may actually be present in the biological sample, thereby resulting in a high possibility of false negatives. In addition, since various substances other than nucleic acids are present in stool, blood and other biological samples, there is also the problem of nucleic acids being extremely susceptible to degradation. Therefore, in order to efficiently recover nucleic acids contained only in relatively small amounts in biological samples such as nucleic acids derived from cancer cells, it is important to be able to stably preserve the nucleic acids until the time of their use in testing procedures by preventing the degradation thereof in biological samples when preparing nucleic acid-containing samples for use in testing from biological samples. In addition, in the case of recovering nucleic acids from biological samples, there is considerable carryover of contaminants (substances other than nucleic acids) inherently contained in biological samples, and there are many cases in which it is difficult to recover nucleic acids of adequate purity. In the case the purity of nucleic acids recovered from a biological sample is inadequate, there is the problem of low reliability of results obtained from testing and analyses carried out using the recovered nucleic acids in the same manner as in the case of poor recovery efficiency.

For example, (1) methods for stably preserving biological samples prior to nucleic acid extraction have been disclosed in which a collected whole blood sample is immediately contacted with a stabilization additive to prevent ex vivo gene induction in the sample and protect the in vivo transcription profile, wherein a detergent, chaotropic salt, ribonuclease inhibitor, chelating agent, mixture thereof, organic solvent or organic reducing agent is used for the stabilization additive (see, for example, Patent Document 1). In addition, (2) a fixative composition for preserving tissues and biological samples has been disclosed that contains one or more alkanol, polyethylene glycol having a molecular weight of 200 to 600, one or more weak organic acids mixed at a concentration of 0.01 to 0.10 moles per liter of the fixative composition and water, and is substantially free of any crosslinking binders (see, for example, Patent Document 2). Differing from formaldehyde solutions and the like routinely used to fix thin sections and other tissue samples for microscopic observation, the use of this fixative composition makes it possible to inhibit denaturation of DNA and RNA during fixation.

On the other hand, methods have also been disclosed for inhibiting carryover of inhibitory substances in a biological sample when extracting and purifying nucleic acids from a stool or other biological sample. For example, (3) a method has been disclosed for enzymatically amplifying nucleic acids comprising washing a test sample with an organic solvent to remove substances that inhibit a nucleic acid enzymatic amplification reaction followed by enzymatically amplifying nucleic acids of cells contained in the test sample (see, for example, Patent Document 3). In addition, (4) a method has been disclosed for removing substances that inhibit nucleic acid amplification contained in a biological sample by treating the biological sample with an acid solution, and preferably an inorganic acid solution (see, for example, Patent Document 4). In addition, since phosphate ions in a reaction solution act in an inhibitive manner in nucleic acid amplification reactions, (5) a method has been disclosed for carrying out a nucleic acid amplification reaction by acidifying a sample to be used in the nucleic acid amplification reaction followed by replacing with a buffer suitable for the nucleic acid amplification reaction in order to remove excess phosphate ions (see, for example, Patent Document 5). Moreover, (6) a method for extracting nucleic acids has been disclosed that comprises a step in which a step for selectively removing acid extracts by mixing a specimen containing nucleic acids such as sputum with an acid such as hydrochloric acid, trichloroacetic acid, acetic acid, phosphoric acid, sulfuric acid or citric acid is preferably carried out two times or more, a step for destroying the cell membrane or cell wall of cells in the specimen, and a step for selectively insolubilizing nucleic acids in the specimen (see, for example, Patent Document 6).

In addition, various microorganisms are present in soil and the like, and in the case of extracting nucleic acids from these microorganisms, examples of methods for removing humic substances contained in the soil have been disclosed, such as (7) a method for directly extracting DNA from microorganisms in soil comprising a first washing step for washing with a weakly acidic aqueous solution containing a compound selected from the group consisting of inorganic acids, organic acids and urea, and a second washing step for washing with an aqueous solution of powdered milk (see, for example, Patent Document 7).

### PRIOR ART DOCUMENTS

### Patent Documents

Patent Document 1: Japanese Unexamined Patent Application Publication (Translation of PCT Application) No. 2004-534731
Patent Document 2: Japanese Unexamined Patent Application Publication (Translation of PCT Application) No. 2008-502913
Patent Document 3: International Patent Publication No. WO 00/08136
Patent Document 4: Japanese Unexamined Patent Application, First Publication No. 2003-159056
Patent Document 5: Japanese Unexamined Patent Application Publication (Translation of PCT Application) No. 2000-516094
Patent Document 6: Japanese Unexamined Patent Application, First Publication No. 2003-267989
Patent Document 7: Japanese Unexamined Patent Application, First Publication No. H10-23895

In the examples described in the aforementioned Patent Document 1, a blood sample prepared by adding a stabilization additive is subjected to centrifugal separation treatment followed by discarding the supernatant, washing the pellet once with water and using the pellet for nucleic acid extraction treatment. In addition, in the examples described in the aforementioned Patent Document 2 as well, nucleic acid extraction is carried out after washing a tissue sample after fixing the tissue. However, in either of these patent documents, there is no description or suggestion whatsoever regarding the effect the type of washing solution used in the washing procedure has on the efficacy of the subsequent nucleic acid extraction.

On the other hand, in the method described in (3) above, the product of washing a stool or other biological sample with an organic solvent is used as is to analyze a nucleic acid amplification reaction and the like. In other words, in the method described in (3) above, since the organic solvent used for washing is carried over to the analysis reaction, there is the problem of a decrease in extraction efficiency caused by carryover of the organic solvent.

In the methods of (4), (5) and (7) above, although inhibitory substances derived from a biological sample are removed by using an acidic solution, since nucleic acids in the biological sample are unstable, the nucleic acids ends up degrading during the washing step, thereby resulting in the problem of inadequate nucleic acid extraction efficiency. Moreover, in the methods of (4) to (7) above, although it is described that the efficiency of a nucleic acid amplification reaction is enhanced by removal of inhibitory substances, there is no description or suggestion whatsoever regarding whether or not nucleic acid extraction efficiency can be improved by treating the biological sample with acid.

### SUMMARY OF THE INVENTION

An object of the present invention is to provide a method of preparing a sample enabling a nucleic acid to be efficiently recovered from a stool or other biological sample without requiring a complicated procedure, and a method of recovering a nucleic acid in a biological sample by using a nucleic acid-containing sample prepared according to that method.

As a result of conducting extensive studies to solve the aforementioned problems, the inventors of the present invention found that a nucleic acid-containing sample having extremely superior nucleic acid extraction efficiency can be prepared by stabilizing nucleic acids in a biological sample by mixing the collected biological sample with a nucleic acid stabilizer, and then washing the stabilized sample with an acidic buffer solution having a pH of 2 to 14 prior to the nucleic acid extraction procedure, thereby leading to completion of the present invention.

Namely, the present invention provides the following.
(1) A method of preparing a nucleic acid-containing sample from a biological sample, comprising:
   (A) mixing a biological sample with a nucleic acid stabilizer to obtain a mixture,
   (B) recovering a solid component from the mixture obtained in (A) to obtain a nucleic acid-containing sample, and
   (C) washing the solid component recovered in (B) using an acidic buffer solution having a pH of 2 to 14.
(2) The method of preparing a nucleic acid-containing sample described in (1) above, wherein the pH of the acidic buffer solution is 3 to 6.
(3) The method of preparing a nucleic acid-containing sample described in (1) or (2) above, wherein the nucleic acid stabilizer is at least one of a water-soluble organic solvent, a protease inhibitor, a polycation and a hypertonic solution.
(4) The method of preparing a nucleic acid-containing sample described in (3) above, wherein the water-soluble organic solvent contains at least one of a water-soluble alcohol, ketone, and an aldehyde.
(5) The method of preparing a nucleic acid-containing sample described in (4) above, wherein the water-soluble alcohol is ethanol, propanol or methanol.
(6) The method of preparing a nucleic acid-containing sample described in (4) above, wherein the ketone is acetone or methyl ethyl ketone.
(7) The method of preparing a nucleic acid-containing sample described in (3) above, wherein the nucleic acid stabilizer is a water-soluble organic solvent, and wherein the concentration of the water-soluble organic solvent in the mixture is 30% or more.
(8) The method of preparing a nucleic acid-containing sample described in (3) above, wherein the nucleic acid stabilizer is a water-soluble organic solvent, and wherein the concentration of the water-soluble organic solvent in the mixture is 0.01% to 30%.
(9) The method of preparing a nucleic acid-containing sample described in (3) above, wherein the protease inhibitor is at least one of a peptide-based protease inhibitor, a reducing agent, a protein denaturing agent, and a chelating agent.
(10) The method of preparing a nucleic acid-containing sample described in (3) above, wherein the protease inhibitor is at least one of AEBSF, aprotinin, bestatin, E-64, leupeptin, pepstatin A, urea, dithiothreitol (DTT) or EDTA.
(11) The method of preparing a nucleic acid-containing sample described (1) above, wherein the polycation is polylysine.
(12) The method of preparing a nucleic acid-containing sample described in (1) above, wherein the acidic buffer solution is a buffer solution selected from the group consisting of an acetic acid/sodium acetate buffer system, a citric acid/sodium hydroxide buffer system and a lactic acid/sodium lactate buffer system.
(13) The method of preparing a nucleic acid-containing sample described in (1) above, wherein the pH of the acidic buffer solution is 3.5 to 5.5.
(14) The method of preparing a nucleic acid-containing sample described in (13) above, wherein the pH of the acidic buffer solution is 4.0 to 5.0.
(15) The method of preparing a nucleic acid-containing sample described in (1) above, wherein the mixture in (A) further comprises a surfactant.
(16) The method of preparing a nucleic acid-containing sample described in (1) above, wherein the mixture in (A) further comprises a colorant.
(17) The method of preparing a nucleic acid-containing sample described in (1) above, wherein the biological sample is stool, blood or urine.
(18) A nucleic acid-containing sample prepared according to the method of preparing a nucleic acid-containing sample described in (1) above.
(19) A method of recovering nucleic acids from a nucleic acid-containing sample prepared from a biological sample using the method of preparing a nucleic acid-containing sample described in (1), comprising:

simultaneously recovering nucleic acids derived from all biological species contained in the biological sample.
(20) A method of recovering nucleic acids from a nucleic acid-containing sample prepared from stool using the method of preparing a nucleic acid-containing sample described in (1) above, comprising:

simultaneously recovering nucleic acids derived from normal intestinal bacterial flora and nucleic acids derived from an organism other than normal intestinal bacterial flora.
(21) The method of recovering a nucleic acid described in (20) above, wherein the organisms other than normal intestinal bacterial flora are mammalian cells.
(22) The method of recovering a nucleic acid described in (19) above, wherein the simultaneous recovering of nucleic acids comprises:
   (a) denaturing protein present in the nucleic acid-containing sample and eluting nucleic acids from cells derived from all biological species contained in the nucleic acid-containing sample, and
   (b) recovering nucleic acids eluted in (a).
(23) The method of recovering a nucleic acid described in (22) above, wherein the simultaneous recovering of nucleic acids further comprises:
   (c) removing the protein denatured in (a)
      wherein (C) is carried out after (a) and before (b).
(24) The method of recovering a nucleic acid described in (23) or (22), wherein the denaturing of protein in (a) is carried out using one or more types of denaturing agents selected from the group consisting of a chaotropic salt, an organic solvent and a surfactant.
(25) The method of recovering a nucleic acid described in (24) above, wherein the organic solvent is phenol.
(26) The method of recovering nucleic acids described in (23) above, wherein the removing of the protein in (c) is carried out using chloroform.
(27) The method of recovering nucleic acids described in (22) above, wherein the recovering of nucleic acid in (b) comprises
   (b1) adsorbing the nucleic acid eluted in (a) to an inorganic support, and
   (b2) eluting the nucleic acid adsorbed in (b1) from the inorganic support.
(28) The method of recovering a nucleic acid described in (22) above, further comprising:
   (d) recovering a solid component from the nucleic acid-containing sample before (a).
(29) A method of analyzing a nucleic acid, comprising analyzing a nucleic acid derived from a mammalian cell by using the nucleic acid recovered from a nucleic acid-containing sample using the method of recovering a nucleic acid described in (20) above.
(30) The method of analyzing a nucleic acid described in (29) above, wherein the mammalian cell is a digestive tract cell.
(31) The method of analyzing a nucleic acid described in (29) above, wherein the mammalian cell is an exfoliated large intestine cell.
(32) The method of analyzing a nucleic acid described in (29) above, wherein the nucleic acid derived from the mammalian cell is a marker indicating a neoplasmic transformation.
(33) The method of analyzing a nucleic acid described in (29) above, wherein the nucleic acid derived from the mammalian cell is a marker indicating an inflammatory digestive tract disease.
(34) The method of analyzing a nucleic acid described in (29) above, wherein the nucleic acid derived from the mammalian cell is a nucleic acid derived from COX-2 gene.

(35) The method of analyzing a nucleic acid described in (29) above, wherein the analysis is one or more types selected from the group consisting of mRNA expression analysis, K-ras gene mutation analysis and DNA methylation analysis.

According to the method of preparing a nucleic acid-containing sample of the present invention, a nucleic acid-containing sample enabling efficient recovery of nucleic acid can be easily prepared from a biological sample. In addition, the method of preparing a nucleic acid-containing sample of the present invention is preferable for preparing biological samples containing comparatively large amounts of contaminants and in which nucleic acids are easily lost as in the manner of stool samples and the like in particular since nucleic acids present in the biological sample are recovered after having been stabilized.

### BRIEF DESCRIPTION OF THE DRAWINGS

FIG. 1 is a drawing showing the results of quantifying RNA recovered from solid components prepared from stool in Example 1.

FIG. 2 is a drawing showing stained images obtained by electrophoresis of RNA recovered in Example 1.

FIG. 3 is a drawing showing the results of quantifying RNA recovered from solid components prepared from stool in Example 2.

FIG. 4 is a drawing showing the amounts of RNA recovered from stool samples in Reference Example 1.

FIG. 5 is a drawing showing the amounts of RNA recovered from stool samples prepared using various concentrations of ethanol solutions in Reference Example 3.

### DETAILED DESCRIPTION OF THE PREFERRED EMBODIMENTS

<Method of Preparing Nucleic Acid-Containing Sample>

The method of preparing a nucleic acid-containing sample of the present invention (to also be referred to as the preparation method of the present invention) is a method of preparing a nucleic acid-containing sample from a biological sample, and is comprised of the following (A) to (C):

(A) mixing a biological sample with a nucleic acid stabilizer to obtain a mixture,

(B) recovering a solid component from the mixture obtained in (A) to obtain a nucleic acid-containing sample, and

(C) washing the solid component recovered in (B) using an acidic buffer solution having a pH of 2 to 14.

In the preparation method of the present invention, since nucleic acids in a biological sample are stabilized in advance by treating the biological sample with a nucleic acid stabilizer prior to nucleic acid extraction treatment, nucleic acids can be efficiently recovered from biological samples containing large amounts of contaminants such as microorganisms and enzymes while minimizing time-based changes of nucleic acids in the biological sample with respect to molecular profiling.

The following provides an explanation of each step.

First, in (A), a biological sample and a nucleic acid stabilizer are mixed to prepare a mixture. In the case the biological sample is a liquid sample such as urine, the nucleic acid stabilizer is added directly to the biological sample and mixed therewith. On the other hand, in the case the biological sample is a sample having a comparatively large solid component such as stool, a nucleic acid stabilizer solution can be prepared by dissolving or diluting the nucleic acid stabilizer in a suitable solvent followed by mixing the nucleic acid stabilizer solution and the biological sample. Furthermore, the biological sample and the nucleic acid stabilizer may also be mixed directly in the case the nucleic acid stabilizer consists of an adequate amount of liquid.

In the description of the present invention and the present application, the term “nucleic acid stabilizer” refers to a compound that has the action and effect of inhibiting degradation of nucleic acids and nucleic acid strand synthesis. In other words, mixing a biological sample with the nucleic acid stabilizer makes it possible to minimize loss of nucleic acids in a biological sample due to degradation and the like and inhibit the synthesis of new nucleic acid strands.

The nucleic acid stabilizer used in the present invention is preferably at least one of a water-soluble organic solvent, a protease inhibitor, a polycation and a hypertonic solution. For example, a solution obtained by dissolving a protease inhibitor, polycation or salt in a water-soluble organic solvent or diluted solution thereof may be mixed with a biological sample.

In the case of mixing a biological sample with a nucleic acid stabilizer solution obtained by dissolving or diluting the nucleic acid stabilizer in a suitable solvent, there are no particular limitations on the solvent used to dissolve or dilute the nucleic acid stabilizer provided it is highly effective for recovering nucleic acids of the present invention, namely prevents degradation and so forth of nucleic acids in a biological sample, stabilizes and preserves nucleic acids, and does not impair the effect of highly efficient recovery of nucleic acids. For example, the solvent may be water or a buffer such as PBS.

In the description of the present invention and present application, a water-soluble organic solvent refers to an organic solvent that is highly soluble in water or can be mixed with water at any arbitrary ratio. Biological samples such as stools normally contain a large amount of moisture, and consequently, by using a water-soluble organic solvent having high solubility in water or which can be mixed with water at any arbitrary ratio for the nucleic acid stabilizer, the biological sample and the nucleic acid stabilizer can be mixed rapidly and higher nucleic acid recovery effects can be obtained.

Although the reason for a water-soluble organic solvent being able to function as a nucleic acid stabilizer is unclear, it is presumed to be the result of the activity of various types of degrading enzymes such as protease, DNase or RNase present in biological samples decreasing considerably since the cellular activity of mammalian cells or microorganisms and the like contained in biological samples decreases considerably due to the dehydrating action possessed by water-soluble organic solvent components, and due to protein denaturing action possessed by water-soluble organic solvent components.

Specific examples of water-soluble organic solvents used as nucleic acid stabilizers include alcohols, ketones and aldehydes, and refer to solvents having a linear structure that are liquids in the vicinity of room temperature, such as a temperature of 15° C. to 40° C. As a result of using a water-soluble organic solvent having a linear structure as an active ingredient, mixing with a biological sample can be carried out more rapidly than in the case of using an organic solvent having a cyclic structure in the manner of benzene and the like as an active ingredient. Since organic solvents having a cyclic structure typically easily separate from water, it is difficult to mix such organic solvents with a biological sample such as stool, thereby making it difficult to obtain high nucleic acid recovery effects. This is because there are many cases in which it is necessary to mix vigorously or apply heat in order to uniformly disperse a biological sample such as a stool even in the case of a solvent that dissolves in water to a certain degree. Furthermore, it is also possible to prepare a mixed solution of an organic solvent and water in advance and mix the mixed solution with a biological sample in order to facilitate mixing between a nucleic acid-containing sample and an organic solvent having a cyclic structure. However, there are many cases in which it is necessary to vigorously mix the organic solvent having a cyclic structure and water or apply heat in order to prepare the mixed solution, thereby making this undesirable.

A water-soluble organic solvent having solubility in water of 12% by weight or more is preferable, a water-soluble organic solvent having solubility in water of 20% by weight or more is more preferable, a water-soluble organic solvent having solubility in water of 90% by weight or more is even more preferable, and a water-soluble organic solvent that can be mixed with water at any arbitrary ratio is particularly preferable for the nucleic acid stabilizer of the present invention. Examples of water-soluble organic solvents that can be mixed with water at any arbitrary ratio include methanol, ethanol, n-propanol, 2-propanol, acetone and formaldehyde.

There are no particular limitations on the water-soluble organic solvent used as a nucleic acid stabilizer of the present invention provided it satisfies the aforementioned definition and is able to demonstrate high nucleic acid recovery effect. Examples of the water-soluble organic solvent include water-soluble alcohols such as methanol, ethanol, propanol, butanol and mercaptoethanol, ketones such as acetone or methyl ethyl ketone (having solubility in water of 90% by weight or more), and aldehydes such as acetoaldehyde (acetyl aldehyde), formaldehyde (formalin), glutaraldehyde, paraformaldehyde or glyoxal. The propanol may be n-propanol or 2-propanol. In addition, the butanol may be 1-butanol (having solubility in water of 20% by weight or more) or 2-butanol (having solubility in water of 12.5% by weight or more). Preferable examples of water-soluble organic solvents used in the present invention include water-soluble alcohols, acetone, methyl ethyl ketone and formaldehyde. This is because these water-soluble organic solvents have sufficiently high solubility in water. Water-soluble alcohols are more preferable from the viewpoints of availability, handling ease and safety, and ethanol and propanol are more preferable. Ethanol is particularly useful for screening examinations such as periodic health examinations since it has the highest degree of safety and is handled easily even in the home.

The water-soluble organic solvent may be mixed directly with a biological sample, or a diluted water-soluble organic solvent obtained by diluting the water-soluble organic solvent with a suitable solvent may be mixed with the biological sample. There are no particular limitations on the concentration of the water-soluble organic solvent in the diluted solution of the water-soluble organic solvent provided it is a concentration that allows the demonstration of high nucleic acid recovery effects, and can be suitably determined in consideration of the type of water-soluble organic solvent and the like. By making the concentration of the water-soluble organic solvent in a diluted solution of the water-soluble organic solvent to be a sufficiently high concentration, in the case of mixing a biological sample with the diluted water-soluble organic solvent solution, the water-soluble organic solvent component is able to rapidly permeate throughout the biological sample, thereby making it possible to rapidly demonstrate high nucleic acid recovery effects.

For example, in the case of using a water-soluble alcohol or ketone, the concentration of the water-soluble organic solvent in a diluted solution of the water-soluble organic solvent is preferably 30% or more, more preferably 50% or more, even more preferably 50% to 80%, and particularly preferably 60% to 70%. The higher the concentration of the water-soluble organic solvent, the greater the degree to which sufficiently high nucleic acid recovery effects can be obtained by using a small amount of sample preparation solution even for a stool sample having a high moisture content.

In addition, in the case of using acetone or methyl ethyl ketone, the concentration of the water-soluble organic solvent in a diluted solution of the water-soluble organic solvent is preferably 30% or more, more preferably 60% or more and even more preferably 80% or more. In the case of using other water-soluble organic solvents such as acetoaldehyde, formaldehyde, glutaraldehyde, paraformaldehyde or glyoxal as active ingredients, the concentration of the water-soluble organic solvent in a diluted solution of the water-soluble organic solvent is preferably 0.01% to 30%, more preferably 0.03% to 10%, and even more preferably 3% to 5%. Aldehydes are able to demonstrate high nucleic acid recovery effects at lower concentrations than alcohols or ketones.

In addition, the water-soluble organic solvent used in the present invention may contain only one type of water-soluble organic solvent or may be a mixed solution of two or more types of water-soluble organic solvents. For example, it may be a mixed solution of two or more types of alcohols or a mixed solution of an alcohol and another type of water-soluble organic solvent. The water-soluble organic solvent is preferably a mixed solution of an alcohol and an acetone in order to further improve high nucleic acid recovery effects.

In addition, in the present invention, a nucleic acid stabilizer having for an active ingredient thereof a protease inhibitor and not an inhibitor of nucleic acid degradation is preferably used for the nucleic acid stabilizer. Normally, nucleic acids present in a biological sample are present in a state of being contained in cells. As a result of proteins and so forth of the cell membrane being subsequently degraded by protease contained in the biological sample, the proteins and the like flow outside the cells through pores formed in the cell membrane, and cell-derived components such as nucleic acids that have flown outside the cells end up being degraded by the action of nucleases present in large amounts in biological samples. Therefore, in the present invention, the use of a protease inhibitor for the nucleic acid stabilizer makes it possible to improve preservation of cell-derived components by effectively inhibiting degradation of cell membrane proteins in biological samples and maintaining cell-derived components such as nucleic acids within cells where there are comparatively small amounts of degrading enzymes and the like.

In the present invention, there are no particular limitations on the protease inhibitor used as nucleic acid stabilizer provided it is able to inhibit the enzyme activity of proteases (enzymes able to catalyst hydrolysis of peptide bonds), and the protease inhibitor may be a proteinase inhibitor or a peptidase inhibitor. In addition, the protease inhibitor may also be that which is able to inhibit serine proteases, that which is able to inhibit cysteine proteases, that which is able to inhibit aspartic proteinases or that which is able to inhibit metalloproteases.

A protease inhibitor can be used for the protease inhibitor used in the present invention that is suitably selected from known protease inhibitors. Examples of protease inhibitors used in the present invention include AEBSF, aprotinin, bestatin, calpain inhibitor I, calpain inhibitor II, chymostatin, 3,4-dichloroisocoumain, E-64, lactacystin, leupeptin, MG-115, MG-132, pepstatin A, PMSF, proteasome inhibitor, TLCK, TPCK and trypsin inhibitor. In addition, combinations of several types of protease inhibitors generally referred to as “protease inhibitor cocktails” can also be used.

In addition, there are no particular limitations on the concentration of the aforementioned protease inhibitor added to a biological sample provided it is an adequate concentration for inhibiting proteases present in a biological sample, and can be suitably determined in consideration of the type of protease inhibitor added, the mixing ratio with the biological sample, and the pH and temperature of the mixture prepared by mixing with the biological sample. Table 1 lists preferable concentrations of each protease inhibitor in a mixture prepared by mixing with a biological sample.

TABLE 1

| Protease inhibitor | Concentration | |
|---|---|---|
| AEBSF | 0.1 to 1.0 | mg/ml |
| Aprotinin | 0.06 to 2 | μg/ml |
| Bestatin | 4 to 400 | μg/ml |
| Calpain inhibitor I | 1 to 100 | μg/ml |
| Calpain inhibitor II | 1 to 100 | μg/ml |
| Chymostatin | 6 to 60 | μg/ml |
| 3,4-dichloroisocoumain | 1 to 43 | μg/ml |
| E-64 | 0.5 to 10 | μg/ml |
| Lactacystin | 0.1 to 10 | μg/ml |
| Leupeptin | 0.1 to 10 | μg/ml |
| MG-115 | 0.1 to 10 | μM |
| MG-132 | 0.1 to 10 | μM |
| Pepstatin A | 0.7 | μg/ml |
| PMSF | 17 to 170 | μg/ml |
| Proteasome inhibitor | 0.1 to 10 | μM |
| TLCK | 37 to 50 | μg/ml |
| TPCK | 70 to 100 | μg/ml |
| Trypsin inhibitor | 10 to 100 | μg/ml |

The protease inhibitor used in the present invention may be a peptide-based protease inhibitor as previously described, a reducing agent, a protein denaturing agent or a chelating agent. Furthermore, in the present invention, a “peptide-based protease inhibitor” refers to a peptide, or modified form thereof, which is able to inhibit protease activity by interacting with protease.

Examples of chelating agents include ethylenediamine tetraacetic acid (EDTA), O,O′-bis(2-aminophenylethylene glycol) ethylenediamine tetraacetic acid (BAPTA), N,N-bis(2-hydroxyethyl) glycine (Bicine), trans-1,2-diaminocyclohexane-ethylenediamine tetraacetic acid (CyDTA), 1,3-diamino-2-hydroxypropane-ethylenediamine tetraacetic acid (DPTA-OH), diethylenetriamine pentaacetic acid (DTPA), ethylenediamine dipropionate (EDDP), ethylenediamine dimethylene phosphonic acid monohydrate (EDDPO), N-(2-hydroxyethyl)ethylenediamine triacetic acid (EDTA-OH), ethylenediamine tetramethylene phosphonic acid (EDTPO), O,O′-bis(2-aminoethyl)ethylene glycol tetraacetic acid (EGTA), N,N-bis(2-hydroxybenzyl)ethylenediamine diacetic acid (HEED), 1,6-hexamethylenediamine tetraacetic acid (HDTA), N-(2-hydroxyethyl)iminodiacetic acid (HIDA), iminodiacetic acid (IDA), 1,2-diaminopropane tetraacetic acid (Methyl-EDTA), nitrilotriacetic acid (NTA), nitrilotripropionate (NTP), nitrilotris(methylene) triphosphonic acid trisodium salt (NTPO), tetrakis(2-pyridylmethyl)ethylenediamine (TPEN) and triethylenetetraamine hexaacetic acid (TTHA).

There are no particular limitations on the concentration of the chelating agent added to a biological sample as protease inhibitor provided it is an adequate concentration for inhibiting protease in the biological sample, and can be suitably determined in consideration of the type of chelating agent and the like. Each chelating agent is added so that the final concentration thereof in a mixture prepared by mixing with a biological sample is preferably 0.1 mM to 1 M.

Examples of reducing agents include dithiothreitol (DTT) and β-mercaptoethanol.

There are no particular limitations on the concentration of the reducing agent added to a biological sample as a protease inhibitor provided it is an adequate concentration for inhibiting protease in the biological sample, and can be suitably determined in consideration of the type of reducing agent and the like. Each chelating agent is added so that the final concentration thereof in a mixture prepared by mixing with a biological sample is preferably 0.1 mM to 1 M.

Examples of protein denaturing agents include urea, guanine and guanidine salts.

There are no particular limitations on the concentration of the protein denaturing agent added to a biological sample as a protease inhibitor provided it is an adequate concentration for inhibiting protease in the biological sample, and can be suitably determined in consideration of the type of protein denaturing agent and the like. Each protein denaturing agent is added so that the final concentration thereof in a mixture prepared by mixing with a biological sample is preferably 0.1 mM to 1 M.

Furthermore, one type of protease inhibitor only may be used or two or more types of protease inhibitors may be used as nucleic acid stabilizer. In addition, a plurality of types of peptide-based protease inhibitors such as AEBSF may also be used in combination, and different types of protease inhibitors may also be used in the manner of a peptide-based protease inhibitor and a chelating agent, or a peptide-based protease inhibitor and a reducing agent.

In the present invention, a polycation is preferably used as a nucleic acid stabilizer. Mixing a biological sample with a polycation makes it possible to effectively reduce nucleic acid degradation and synthesis reactions attributable to contaminants contained in the biological sample. In addition, although large amounts of substances that inhibit reactions used in nucleic acid analyses such as nucleic acid strand extension reactions are contained in biological samples, mixing the biological sample with a polycation also makes it possible to reduce the inhibitory action of these inhibitory substances.

Furthermore, in the description of the present application, an inhibitory substance refers to a substance that demonstrates an inhibitory action on enzyme reactions that use a nucleic acid as substrate. There are no particular limitations on these enzyme reactions provided they are enzyme reactions that use nucleic acid as substrate, and examples thereof include enzyme reactions typically used in nucleic acid analyses, such as reverse transcription reactions or base chain extension reactions. Here, a base chain extension reaction refers to a base chain extension reaction by a polymerase or ligase. Examples of base chain extension reactions by polymerase include polymerase chain reaction (PCR), real-time PCR and standard displacement amplification (SDA). Examples of base chain extension reactions by ligase include ligase chain reaction (LCR).

Specific examples of these inhibitory substances include bile acids and bile salts.

In the description of the present invention and present application, a polycation refers to a polymeric compound or salt thereof having a repeating structure containing cationic functional groups. An example of a cationic functional group is an amino group. Specific examples of polycations include polypeptides having a cationic functional group in a side chain thereof such as polylysine indicated in the following formula (1), and polymers obtained by polymerizing a monomer containing a cationic functional group in a side chain thereof such as polyacrylamide. Furthermore, although these polypeptides and polymers are only required to be electrically positive in terms of their overall molecular charge, and the side chains of all of their repeating units (amino acids or monomers) are not required to have cationic functional groups, the side chains of all repeating units preferably have cationic functional groups. Specific examples of such polycations include polylysine and polyacrylamide as well as polyvinylamine, polyallylamine, polyethylamine, polymethacrylamine, polyvinylmethyl imidazole, polyvinyl pyridine, polyarginine, chitosan, 1,5-dimethyl-1,5-diazaundecamethylene-polymethobromide, poly(2-dimethylaminoethyl (meth)acrylate) poly(2-diethylaminoethyl (meth)acrylate), poly(2-trimethylammoniumethyl (meth)acrylate) polydimethylaminomethyl styrene, polytrimethylammoniummethyl styrene, polyornithine and polyhistidine. In the present invention, the polycation is preferably polylysine or polyacrylamide, and more preferably polylysine. Furthermore, one type of polycation may be used or two or more types of polycations may be used for the nucleic acid stabilizer used in the present invention.

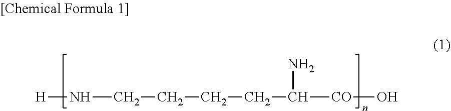

There are no particular limitations on the concentration of polycation added as nucleic acid stabilizer to a biological sample provided it is an adequate concentration for obtaining the effect of reducing the inhibitory action of inhibitory substances contained in the biological sample (inhibitory action reducing effect), and can be suitably determined in consideration of the type of polycation, type of nucleic acid-containing sample, pH of the sample preparation solution, and mixing ratio between the sample preparation solution and the nucleic acid-containing sample. For example, in the case of containing polylysine as the polycation, the concentration of polylysine in the sample preparation solution is preferably 0.01 m % by weight to 1.0 m % by weight, more preferably 0.0125 m % by weight to 0.8 m % by weight and even more preferably 0.05 m % by weight to 0.4 m % by weight. Furthermore, in the description of the present application, the term “m % by weight” refers to “×10⁻³% by weight”.

Moreover, a hypertonic solution may also be used for the nucleic acid stabilizer. Although the reason why a hypertonic solution is able to function as a nucleic acid stabilizer is unclear, since various types of degrading enzymes end up precipitating as a result of salting out, it is presumed that this effect is obtained as a result of the activities of various types of degrading enzymes such as proteases, DNases or RNases in stool decreasing considerably due to cellular activities of mammalian cells and bacteria such as normal intestinal bacterial flora decreasing significantly resulting in inhibition of changes over time due to the dehydrating action of highly concentrated salt components, as well as the salt concentration deviating from the optimum salt concentration.

Salts contained as active ingredients of hypertonic solutions can be used by suitably selecting from salts normally used when preparing or analyzing biological samples. For example, the salt may be a chloride, sulfate or acetate. In addition, one type of salt may be used or two or more types of salts may be used in combination for the active ingredient. The hypertonic solution used in the present invention preferably contains as an active ingredient thereof one or more types of salts selected from the group consisting of sodium chloride, potassium chloride, ammonium sulfate, ammonium bisulfate, ammonium chloride, ammonium acetate, cesium sulfate, cadmium sulfate, cesium iron(II) sulfate, chromium(III) sulfate, cobalt(II) sulfate, copper(II) sulfate, lithium chloride, lithium acetate, lithium sulfate, magnesium sulfate, manganese sulfate, sodium sulfide, sodium acetate, sodium sulfate, zinc chloride, zinc acetate and zinc sulfate. In terms of availability, handling ease and stability in particular, the hypertonic solution preferably contains sodium chloride and/or ammonium sulfate. Sodium chloride is particularly useful for screening examinations such as periodic health examinations since it has the highest degree of safety and can be handled easily even in the home.

There are no particular limitations on the concentration (also referred to as “salinity”) of the salt contained as active ingredient in the hypertonic solution provided it is an adequate concentration that allows the hypertonic solution to function as a nucleic acid stabilizer, and can be suitably determined in consideration of the types of salt and solvent used. Furthermore, the upper limit value of salinity for each of the salts is the saturated concentration. On the other hand, although differing according to the type of salt used, the lower limit value of salinity can be determined experimentally by a person with ordinary skill in the art.

For example, the lower limit value of salinity can be determined in the manner described below. First, a plurality of concentrations of salt solutions having concentrations equal to or less than the saturated concentration are prepared, and nucleic acids are recovered from stool samples that have been immersed in these salt solutions for a prescribed amount of time. The minimum salinity value in the case the amount of recovered nucleic acid is greater than the amount of nucleic acid recovered from stool not treated with the salt solutions can be taken to be the lower limit value of salinity of the salt contained as active ingredient. In addition, in the case of using stool as a biological sample, for example, a bacterial culture or a mixed solution of a mammalian cell culture and a bacterial culture can be used as a pseudo stool sample instead of stool.

In the present invention, in order to obtain higher nucleic acid stabilization effects, the salinity of the hypertonic solution is preferably one-half or more of the saturated concentration of the salt used as active ingredient, more preferably ⅘ or more of the saturated concentration, even more preferably nearly equal to the saturated concentration, and particularly preferably substantially equal to the saturated concentration regardless of the type of salt. As a result of making the salinity to be of a sufficiently high concentration, in the case of mixing a stool with the hypertonic solution, the components thereof are able to rapidly permeate into the stool and rapidly stabilize nucleic acids. In addition, the use of high salinity makes it possible to demonstrate adequate effects even in the case of using a small amount of hypertonic solution for a stool sample having a high moisture content. Furthermore, a “solution having a concentration that is ½ or more the saturated concentration” or a “solution having a concentration that is ⅘ or more the saturated concentration” can be prepared by suitably diluting a saturated solution prepared in accordance with routine methods with a solvent.

For example, in the case of using sodium chloride as an active ingredient, salinity is preferably 13% (wt/wt) or more, more preferably 20% (wt/wt) or more, even more preferably 26% (wt/wt) or more, and particularly preferably a concentration within the range of 26% (wt/wt) to the saturated concentration. In the case of using ammonium sulfate, salinity is preferably 20% (wt/wt) or more, more preferably 30% (wt/wt) or more, and even more preferably 30% to 46% (wt/wt).

Examples of biological samples used in the preparation method of the present invention include stool, urine, blood, cerebrospinal fluid, lymph, sputum, saliva, sperm, bile, pancreatic fluid, ascites, exudate, amniotic fluid, gastrointestinal lavage fluid, pulmonary lavage fluid, bronchial lavage fluid and urinary bladder lavage fluid. Cultures of cultured cells and the like may also be used. The biological sample used in the preparation method of the present invention is particularly preferably stool, blood or urine. In addition, although there are no particular limitations on the biological sample provided it is collected from a living organism, it is preferably derived from a mammal, and more preferably derived from a human. For example, although a biological sample collected from a human for the purpose of a periodic health examination or diagnosis and the like is preferable, it may also be a biological sample collected from a domestic or wild animal. In addition, although the biological sample may have been stored for a fixed period of time after collection, it is preferably used immediately after collection. In the case the biological sample is stool, although the stool used in the preparation method of the present invention is preferably that obtained immediately after voiding, it may also be that for which time has elapsed after voiding.

There are no particular limitations on the amount of biological sample used in the preparation method of the present invention, and can be suitably determined in consideration of, for example, the method used to analyze nucleic acids recovered from the biological sample. In the case of stool, for example, the amount is preferably 10 mg to 1 g. If the amount of stool is excessively large, the collection procedure becomes bothersome and the stool container becomes larger, thereby resulting in the risk of a decrease in handling ease and the like. Conversely, in the case the amount of stool is excessively small, the number of mammalian cells such as exfoliated large intestine cells contained in the stool becomes excessively small, thereby resulting in the risk of being unable to recover the required amount of nucleic acid and causing a decrease in accuracy of the target nucleic acid analysis. In addition, since stool is heterogeneous, or in other words since a wide range of various components are non-uniformly present in stool, the biological sample is preferably collected from a wide range of the stool at the time of collection in order to avoid the effects of localization of mammalian cells.

In the case of a biological sample such as stool having a high solid content, although there are no particular limitations on the volume of the nucleic acid stabilizer solution (or nucleic acid stabilizer) mixed with the collected biological sample, the mixing ratio between the biological sample and the nucleic acid stabilizer solution is preferably such that the volume of the nucleic acid stabilizer solution is 1 or more with respect to the volume of the biological sample. This is because the use of the nucleic acid stabilizer solution in an amount equal to or greater than the amount of the biological sample enables the nucleic acid stabilizer solution to permeate through the entire surface of the biological sample and allows the nucleic acid stabilizer to adequately act on the biological sample. In particular, by mixing the nucleic acid stabilizer solution in an amount equal to or greater than 5 times the amount of the biological sample, the biological sample is able to rapidly and effectively disperse in the nucleic acid stabilizer solution, and the effect of decreasing the concentration of the nucleic acid stabilizer caused by moisture contained in the biological sample can be reduced. On the other hand, there are many cases in which a comparatively small amount for the total amount of the mixture of the biological sample and the nucleic acid stabilizer solution is preferable in terms of handling ease. For example, in the case of using stool, although the stool can be preliminarily collected in a stool collection container containing the nucleic acid stabilizer solution to prepare a mixture thereof in the container, in this case, if the stool and nucleic acid stabilizer solution are present in equal amounts, the stool collection container containing the nucleic acid stabilizer solution can be reduced in weight and size. In this manner, in order to improve handling ease of the biological sample and the resulting mixture as well as dispersibility of the biological sample in the nucleic acid stabilizer solution in the proper balance, in the case the biological sample is stool, the mixing ratio of the biological sample to the nucleic acid stabilizer solution is preferably 1:1 to 1:20, more preferably 1:3 to 1:10, and even more preferably about 1:5.

Mixing of the biological sample and the nucleic acid stabilizer solution (or nucleic acid stabilizer) in step (A) may be carried out by immersing the biological sample in the nucleic acid stabilizer solution without using an extraordinary stirring procedure. This is because, since the nucleic acid stabilizer and solution thereof used in the present invention acclimate extremely easily even to biological samples such as stool having a high moisture content, depending on the amount and state of the biological sample being mixed, adequately high nucleic acid recovery effects are demonstrated simply by immersing the biological sample in the nucleic acid stabilizer solution to adequately allow the nucleic acid stabilizer solution to permeate the biological sample even in the case of not employing an extraordinary stirring procedure.

Mixing of the biological sample and the nucleic acid stabilizer solution (or nucleic acid stabilizer) may also be carried out by adding and immersing the biological sample to the nucleic acid stabilizer solution followed by stirring. Stirring enables the biological sample to be adequately dispersed and suspended in the nucleic acid stabilizer solution. In the case of preparing a suspension by adding the biological sample and stirring the nucleic acid stabilizer solution, stirring is preferably carried out rapidly. This is because rapidly dispersing the biological sample in the nucleic acid stabilizer solution enables the nucleic acid stabilizer to rapidly permeate cells in the biological sample and act on contaminants in the biological sample, thereby allowing the obtaining of even higher nucleic acid recovery effects.

Furthermore, there are no particular limitations on the method used to prepare a suspension by mixing the biological sample and the nucleic acid stabilizer solution provided it is a method that consists of mixing by a physical method. For example, a collected biological sample may be placed in a sealable container preliminarily containing the nucleic acid stabilizer solution and sealed followed by mixing by vertically inverting the container or by shaking such as by vortex mixing. In addition, the biological sample and the nucleic acid stabilizer solution may be mixed in the presence of mixing particles. Since mixing is able to be carried out rapidly, methods using a shaker or mixing particles are preferable. In particular, the use of a collection container preliminarily containing mixing particles enables rapid mixing in an environment such as the home where there are no special apparatuses.

There are no particular limitations on the mixing particles provided they are of a composition that does not impair the nucleic acid recovery effects of the nucleic acid stabilizer solution, and have hardness and specific gravity that enable the biological sample to be rapidly dispersed in the nucleic acid stabilizer solution as a result of colliding with stool or other biological sample, and the particles may be composed of one type of material or composed of two or more types of materials. Examples of materials used for these mixing particles include glass, ceramics, plastic, latex and metal. In addition, the mixing particles may be magnetic particles or non-magnetic particles.

In addition, arbitrary components other than the nucleic acid stabilizer may be added to the mixture prepared in step (A) provided they do not impair the high nucleic acid recovery effects of the nucleic acid stabilizer. For example, a chaotropic salt or surfactant may be added. The addition of a chaotropic salt or surfactant is able to effectively inhibit cellular activity as well as enzyme activity of various types of degrading enzymes contained in a biological sample. Examples of chaotropic salts that can be mixed with the biological sample together with the nucleic acid stabilizer include guanidine chloride, guanidine isothiocyanate, sodium iodide, sodium perchlorate and sodium trichloroacetate. The surfactant that can be mixed with the biological sample together with the nucleic acid stabilizer is preferably a nonionic surfactant. Examples of these nonionic surfactants include Tween80, 3-[3-cholamidopropyldimethylammonio]-1-propanesulfonate (CHAPS), Triton X-100 and Tween20. There are no particular limitations on the concentration of the chaotropic salt or surfactant provided it is a concentration that allows the obtaining of high nucleic acid recovery effects, and can be suitably determined in consideration of the amount of biological sample and the methods used for the subsequent nucleic acid recovery and analysis.

In addition, a suitable colorant may also be added to the mixture prepared in step (A). The addition of a colorant to the mixture allows the obtaining of effects such as prevention of accidental swallowing and moderating the color of the biological material. The colorant is preferably a coloring material used as a food additive and is preferably blue or green and the like. Examples of colorants include fast green FCF (Green No. 3), brilliant blue FCF (Blue No. 1) and indigo carmine (Blue No. 2). In addition, a plurality of colorants may be added as a mixture or a single colorant may be added alone.

In addition, the high nucleic acid recovery effects of the nucleic acid stabilizer are not affected by temperature conditions in particular as long as an adequate amount of the nucleic acid stabilizer is present in the mixture. Thus, the preparation method of the present invention is able to inhibit loss of nucleic acids in a biological sample at a temperature at which collection of stool or other biological samples is carried out, namely even in the case the biological sample is collected at room temperature. In addition, nucleic acids in the mixture prepared in step (A) can be stably preserved even in the case the mixture is stored or transported at room temperature until treatment of the subsequent step (B) and beyond is carried out. However, in the case of using a water-soluble organic solvent for the nucleic acid stabilizer, the mixture is preferably stored at 50° C. or lower. This is because storing the mixture under high temperature conditions for a long period of time has the risk of causing the concentration of water-soluble organic solvent in the mixture to decrease below an adequate concentration for demonstrating high nucleic acid recovery effects due to evaporation and the like.

In addition, the mixture obtained in step (A) enables nucleic acids, and particularly RNA readily susceptible to degradation, to be stably preserved at room temperature for a comparatively long period of time while inhibiting degradation thereof due to the presence of the nucleic acid stabilizer. Consequently, in cases in which the location and time during which a biological sample is collected are different from the location and time at which nucleic acid extraction and analysis procedures are carried out or in the case in which a large number of samples must be processed, as in the case of health examinations and other screening examinations, after preparing the mixture by carrying out step (A) at the location where the biological samples are collected, the mixture is stored and transported in a mixed state until the time or location where the nucleic acid extraction and analysis procedures are carried out, and steps (B) and (C) are preferably carried out immediately prior to the nucleic acid extraction procedure.

Next, step (B) is carried out in which a solid component is recovered from the mixture obtained in step (A). In the present invention, nucleic acids contained in a biological sample are stabilized by a nucleic acid stabilizer while contained within cells. Consequently, the solid component that includes cellular components is used as a nucleic acid-containing sample derived from the biological sample.

There are no particular limitations on the method used to recover the solid component from the mixture, and any known method used when recovering solid components from suspensions may be used. For example, the mixture may be centrifuged followed by removal of the supernatant and recovery of the precipitate, or the mixture may be filtered using a filter having a suitable pore size followed by recovering the solid component remaining on the filter surface.

There are no particular limitations on the pore size of the filter used in filtration provided it is of a size that allows only liquid components to pass through, and can be used after suitably selecting from filters having a pore size ordinarily used in the relevant field. For example, in the case of using a biological sample such as urine having a comparatively small amount of solid components, a filter having a comparatively small pore size is used preferably. On the other hand, in the case of using a biological sample such as blood or urine having a comparatively large amount of solid components, since the use of a filter having an excessively small pore size results in the occurrence of clogging, a filter having a comparatively large pore size is used preferably.

Subsequently, the solid component recovered in step (B) is washed using a buffer solution having a pH of 2 to 7.5 in step (C). If an excess of nucleic acid stabilizer is transferred to the extracted and purified nucleic acids, it inhibits nucleic acid analysis and makes accurate analysis difficult, particularly during nucleic acid analyses using a nucleic acid strand extension reaction. In the present invention, since excess nucleic acid stabilizer is removed from the solid component by the washing treatment of step (C), this transfer of nucleic acid stabilizer to extracted nucleic acids from the solid component (nucleic acid-containing sample) can be reduced considerably. Since an especially large amount of nucleic acid stabilizer is transferred in the case of using a bulky biological sample such as stool in particular, although extraction efficiency decreases considerably if extraction is carried out without carrying out a washing step, by carrying out the washing indicated in step (C), a nucleic acid-containing sample can be prepared having superior nucleic acid extraction efficiency.

In the present invention, a buffer solution is preferably used to wash the solid component. This is because a buffer solution is able to inhibit fluctuations in pH in the solid component during washing. In particular, the buffer solution used in step (C) is preferably an acidic buffer solution having buffering action such that the pH is maintained within the range of 2 or higher. The use of an acidic buffer solution for the buffer solution makes it possible to prepare a nucleic acid-containing sample having higher nucleic acid extraction effects than in the case of using simply water or a neutral buffer solution. This is presumed to be because an acidic buffer solution is able to more effectively inhibit nucleic acid hydrolysis by holding the solid component under acidic conditions. Furthermore, a “nucleic acid-containing sample having high nucleic acid extraction effects” refers to a nucleic acid that can be extracted with high efficiency in the case of extracting the nucleic acid from the nucleic acid-containing sample.

In the present invention, the pH of the acidic buffer solution used to wash the solid component is preferably 2 to 6.5, more preferably 3 to 6, even more preferably 3.5 to 5.5, and particularly preferably 4.0 to 5.0.

The acidic buffer solution used in step (C) is preferably a sample preparation solution that contains an organic acid and a conjugate base of the organic acid and demonstrates buffering action by means of the organic acid and the conjugate base thereof. In particular, the acidic buffer solution is preferably a buffer solution selected from the group consisting of a citric acid/sodium hydroxide buffer system, lactic acid/sodium lactate buffer system and acetic acid/sodium acetate buffer system. These buffer solutions can be prepared by, for example, adjusting to a suitable pH by adding an organic acid and an alkaline metal salt or alkaline earth metal salt of the organic acid to water or a suitable solvent. In addition, pH may be adjusted using an hydroxide of an alkaline metal or alkaline earth method after having added an organic acid to water or suitable solvent.

In addition, the acidic buffer solution used in step (C) may also be a solution containing both an organic acid and an inorganic acid that has suitable buffering action. This acidic buffer solution may be a buffer system having buffering action in the acidic range, such as a glycine/HCl buffer system, sodium cacodylate/HCl buffer system or potassium hydrogen phthalate/HCl buffer system.

Furthermore, in the present invention, the pH of the acidic buffer solution is the value obtained by measuring with a pH meter using the glass electrode method for the measuring principle thereof (such as that manufactured by DKK-Toa) after calibrating with a phthalate pH standard and neutral phosphate pH standard.

In this manner, according to the preparation method of the present invention, nucleic acids contained in a biological sample are stabilized, the amount of nucleic acid stabilizer transferred from the biological sample is considerably reduced, and a nucleic acid-containing sample having superior nucleic acid extraction efficiency (nucleic acid recovery efficiency) can be easily prepared. Namely, the use of a nucleic acid-containing sample prepared according to the preparation method of the present invention (to also be referred to as the nucleic acid-containing sample of the present invention) can be expected to contribute to the early detection and diagnosis of various symptoms and diseases, observation of the course of treatment as well as pathological research on other abnormal states as a result of enabling nucleic acids in a biological sample to be analyzed with high sensitivity and high accuracy.

In particular, the nucleic acid-containing sample of the present invention is extremely preferable as a sample used to analyze nucleic acids only contained in comparatively small amounts in biological samples. This is because, although the reliability of nucleic acid analysis is generally susceptible to the effects of the efficiency at which nucleic acids are extracted from a biological sample in cases in which target nucleic acids targeted for analysis are only contained in trace amounts in the biological sample, a nucleic acid-containing sample prepared according to the preparation method of the present invention has extremely superior nucleic acid extraction efficiency. More specifically, the nucleic acid-containing sample of the present invention is extremely preferable as a sample for analysis of nucleic acids derived from cancer cells or infectious disease pathogens as well as analysis of nucleic acids derived from mammalian cells present in stool.

The nucleic acid-containing sample of the present invention enables recovery of nucleic acids and analysis of the resulting nucleic acids in the same manner as other samples containing nucleic acids. There are no particular limitations on the method used to recover and analyze nucleic acids from the nucleic acid-containing sample of the present invention, and can be used by suitably selecting from known recovery methods and analysis methods. In addition, recovery of nucleic acids from the nucleic acid-containing sample of the present invention can also be carried out using a commercially available kit such as a nucleic acid extraction kit.

Furthermore, depending on the subsequent nucleic acid analysis method, nucleic acids are not required to be recovered from the nucleic acid-containing sample of the present invention. More specifically, the nucleic acid-containing sample (solid component) of the present invention may be suspended in a buffer solution and the like preferably used in nucleic acid analysis methods, nucleic acids may be extracted into the buffer solution by adding and mixing an elution buffer such as PBS containing a proteinase such as proteinase K to the resulting suspension, and the resulting supernatant may be used directly in an analysis reaction.

<Nucleic Acid Recovery Method>

In the case of recovering nucleic acids from the nucleic acid-containing sample of the present invention, nucleic acids derived from all biological species contained in the nucleic acid-containing sample, namely nucleic acids derived from all biological species contained in a biological sample, are preferably recovered simultaneously. Even in the case of recovering nucleic acids derived from biological species only contained in comparatively small amounts in a biological sample for the purpose of analysis, simultaneous recovery of nucleic acids derived from all biological species makes it possible to more greatly enhance nucleic acid recovery efficiency than in the case of only recovering nucleic acids derived from a target biological species since the nucleic acids derived from other biological species function as carriers.

For example, in the case of analyzing nucleic acids derived from mammalian cells, such as exfoliated large intestine cells, contained in trace amounts in stool, simultaneously recovering the mammalian cell-derived nucleic acids along with nucleic acids derived from normal intestinal bacterial flora containing in large amounts in stool from the nucleic acid-containing sample of the present invention prepared from the stool makes it possible to efficiently extract and recover the mammalian cell-derived nucleic acids.

Furthermore, normal intestinal bacterial flora refers to bacterial cells present in comparatively large numbers in stool which are normal flora that normally inhabit the intestines of humans and other animals. Examples of normal intestinal bacterial flora include obligate anaerobic bacteria such as Bacteroides species, Eubacterium species, Bifidobacterium species or Clostridium species, and facultative anaerobic bacteria such as Escherichia species, Enterobacter species, Klebsiella species, Citrobacter species or Enterococcus species.

Carrying out nucleic acid analysis using nucleic acids recovered in this manner makes it possible to detect specific disease markers such as those for colon cancer with extremely high sensitivity and accuracy. Furthermore, nucleic acid recovered from the nucleic acid-containing sample of the present invention may be DNA, RNA or both DNA and RNA.

For example, nucleic acids can be recovered from the nucleic acid-containing sample of the present invention by denaturing protein in the nucleic acid-containing sample of the present invention and eluting nucleic acids from cell derived from all biological species contained in the nucleic acid-containing sample in step (a), followed by recovering the eluted nucleic acids in step (b).

Denaturation of protein in the nucleic acid-containing sample in step (a) can be carried out with a known method. For example, protein in the nucleic acid-containing sample can be denatured by adding a compound normally used as a protein denaturing agent, such as a chaotropic salt, organic solvent or surfactant, to the nucleic acid-containing sample. The same chaotropic salts and surfactants listed as examples of chaotropic salts and surfactants able to be added to a biological sample in step (A) of the preparation method of the present invention can be used for the chaotropic salt or surfactant able to be added to the nucleic acid-containing sample in step (a). Phenol is a preferable example of an organic solvent. The phenol may be neutral or acidic. In the case of using an acidic phenol, RNA can be extracted into an aqueous layer more selectively than DNA. Furthermore, in the case of adding a chaotropic salt, organic solvent or surfactant and the like to the nucleic acid-containing sample in step (a), one type of compound may be added or two or more types of compounds may be added.

Furthermore, although a protein denaturing agent such as a chaotropic salt may be added directly to the nucleic acid-containing sample of the present invention (solid component obtained after washing), the protein denaturing agent is preferably added after having first suspended in a suitable chemical agent for elution. In the case of recovering DNA, a phosphate buffer or TRIS buffer, for example, can be used for the elution chemical agent. The elution chemical agent is preferably a chemical agent that causes DNase to be deactivated by high-pressure steam sterilization and the like, and is more preferably a chemical agent that contains a proteinase such as proteinase K. On the other hand, in the case of recovering RNA, although a citrate buffer, for example, can be used for the elution chemical agent, since RNA is a substance that is extremely susceptible to degradation, a buffer containing an RNase inhibitor such as guanidine thiocyanate or guanidine hydrochloride is used preferably.

Protein that has been denatured in step (a) may be removed in step (c) after step (a) prior to carrying out step (b). Preliminarily removing denatured protein prior to recovering nucleic acid makes it possible to improve the quality of the recovered nucleic acid. Removal of protein in step (c) can be carried out with a known method. For example, denatured protein can be removed by precipitating denatured protein by centrifugation and recovering only the resulting supernatant. In addition, denatured protein can be removed more completely than in the case of simply centrifuging by adding chloroform, centrifuging after adequately stirring and mixing with a vortex mixer and the like to precipitate the denatured protein, and then recovering only the supernatant.

Recovery of nucleic acid eluted in step (b) can be carried out with a known method such as ethanol precipitation or cesium chloride ultracentrifugation. In addition, nucleic acid can be recovered by adsorbing nucleic acid eluted in step (a) onto an inorganic support in step (b1) followed by eluting the nucleic acid adsorbed in step (b1) from the inorganic support in step (b2). A known inorganic support capable of adsorbing nucleic acid can be used for the inorganic support used to adsorb nucleic acid in step (b1). In addition, there are no particular limitations on the form of the inorganic support, and it may be in the form of particles or a film. Examples of the inorganic support include silica-containing particles (beads) such as those composed of silica gel, siliceous oxide, glass or diatomaceous earth, and porous films such as those composed of nylon, polycarbonate, polyacrylate or nitrocellulose. A solvent normally used to elute nucleic acids from these known inorganic supports can be suitably used for the solvent used to elute the nucleic acid adsorbed in step (b2) in consideration of the type of nucleic acid recovered, the subsequent nucleic acid analysis method and the like. Purified water is particularly preferable for use as the elution solvent. Furthermore, the inorganic substrate adsorbed with nucleic acid is preferably washed using a suitable washing buffer after step (b1) but prior to step (b2).

Nucleic acid recovered from the nucleic acid-containing sample of the present invention can be analyzed using a known nucleic acid analysis method. Examples of nucleic acid analysis methods include methods used to quantify nucleic acid and methods used to detect a specific base sequence region using PCR and the like. In addition, in the case of recovering RNA, cDNA obtained by synthesizing the cDNA by a reverse transcription reaction from the RNA can be used in analysis in the same manner as DNA. In the case of using DNA recovered from a nucleic acid-containing sample, for example, a mutation analysis or epigenetic variation analysis can be carried out on the DNA. Examples of mutation analyses include an analysis of base insertion, deletion, substitution, duplication or inversion. In addition, examples of an epigenetic variation analysis include an analysis of methylation or demethylation. In addition, the onset of cancer can be investigated by detecting the presence or absence of a genetic mutation such as a base sequence region containing a microsatellite. On the other hand, in the case of using recovered RNA, mutations such as an insertion, deletion, substitution, duplication, inversion or splicing variant (isoform) can be detected on the RNA. In addition, functional RNA analyses (non-coding RNA) or analyses of, for example, transfer RNA (tRNA), ribosomal RNA (rRNA) or microRNA (miRNA) cap, also be carried out. In addition, an expressed amount of RNA can also be detected and analyzed. Analysis of expression of mRNA, analysis of mutation of K-ras gene and analysis of DNA methylation are carried out particularly preferably. Furthermore, these analyses can be carried out according to methods known in the relevant field. In addition, commercially available analysis kits, such as K-ras gene mutation analysis kits or methylation detection kits, may also be used.

The analysis method is preferably used in analyses for the purpose of detecting markers indicating a neoplasmic transformation or markers indicating an inflammatory digestive tract disease in particular. For example, examples of markers indicating a neoplasmic transformation include known cancer markers such as carcinoembryonic antigen (CEA) or sialosyl-Tn (STN) antigen, and those indicating the presence of mutations such as mutations of APC gene, p53 gene or K-ras gene. In addition, detection of methylation of genes such as pit, hMLHI, MGMT, p14, APC, E-cadherin, ESR1 or SFRP2 is also useful as a diagnostic marker for intestinal diseases (see, for example, “A CpG island hypermethylation profile of primary colorectal carcinomas and colon cancer cell lines”, Molecular Cancer, 2004, Vol. 3, Chapter 28). In addition, DNA derived from Helicobacter pylori present in stool samples has been previously reported to be able to be used as a gastric cancer marker (see, for example, Nilsson, at al., Journal of Clinical Microbiology, 2004, Vol. 42, No. 8, pp. 3781-8). On the other hand, nucleic acid derived from Cox-2 gene is an example of a marker indicating an inflammatory digestive tract disease. Furthermore, Cox-2 gene-derived nucleic acid is also used as a marker that indicates neoplasmic transformation.

### EXAMPLES

Although the following provides a more detailed explanation of the present invention by indicating examples thereof, the present invention is not limited to the following examples. Furthermore, the term “%” refers to “% by volume” unless specifically indicated otherwise. In addition, Caco-2 cells used as cultured cells were cultured in accordance with ordinary methods.

### Example 1

Nucleic acid-containing samples were prepared from stool according to the preparation method of the present invention using an 80% ethanol solution as nucleic acid stabilizer.

First, 1 g aliquots of a stool sample collected from a healthy subject were respectively placed in six 15 mL polypropylene tubes. 10 mL aliquots of an 80% ethanol solution were respectively added to three of the tubes, the stool was adequately dispersed therein, and the resulting mixtures were allowed to stand undisturbed for 3 hours at 25° C. (stabilization treatment). After allowing to stand undisturbed, the mixtures were centrifuged followed by removal of the supernatant and recovery of the solid component (stabilized tubes). On the other hand, the remaining three tubes were centrifuged immediately without being treated followed by removal of the supernatant and recovery of the solid component (non-stabilized tubes).

A washing step as described below was carried out on the solid components. First, 10 mL of citric acid/sodium hydroxide buffer (0.1 M, pH 5) were dispensed into one of the three stabilized tubes, while 10 rat of PBS (phosphate buffered saline, pH 7) were dispensed into another of the tubes followed by mixing well for 1 minute, re-centrifuging and recovering the solid components. The washing step was not carried out on the remaining tube. 10 mL of citric acid/sodium hydroxide buffer were similarly dispensed into one of the three non-stabilized tubes, 10 mL of PBS were dispensed into another of the tubes, and the washing step was not carried out on the remaining tube.

RNA was recovered from each of the resulting solid components.

More specifically, a phenol mixture known as “Trizol” (Invitrogen) was added to the resulting solid components followed by mixing well using a vortex mixer, adding chloroform, again mixing well using a vortex mixer, and centrifuging at 12,000×g for 20 minutes at 4° C. The supernatant obtained by centrifugation (aqueous layer) was applied to an RNA recovery column (RNeasy Midi Kit, Qiagen), and RNA was recovered by carrying out a washing procedure and an RNA elution procedure on the RNA recovery column in accordance with the protocol provided.

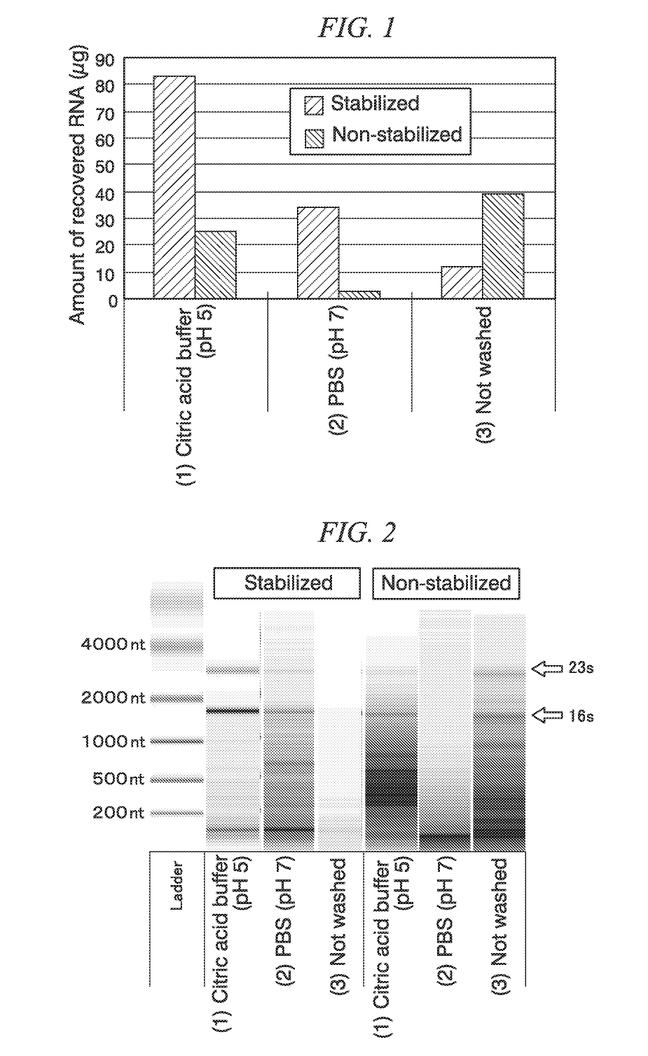

The recovered RNA was quantified using Nanodrop (Nanodrop Products). The results are shown in Table 2 and FIG. 1. In Table 2, “citric acid buffer” refers to citric acid/sodium hydroxide buffer (0.1 M). As a result, in the case of not having stabilized nucleic acids with nucleic acid stabilizer, the amount of RNA recovered decreased more as result of carrying out the washing step than in the case of not carrying out the washing step, thereby suggesting that loss of nucleic acids due to degradation and the like is promoted by the washing step. In addition, even in the case of having stabilized nucleic acids with nucleic acid stabilizer, the amount of RNA recovered decreased more than in the case of having not added a nucleic acid stabilizer when not carrying out the washing step, thereby suggesting that extraction of nucleic acid is inhibited by contaminants present in the stool. In contrast, in the case of having carried out the washing step after having stabilized nucleic acid with nucleic acid stabilizer, the amount of RNA recovered was higher than in the case of not carrying out the washing step. In the case of washing with the citric acid buffer having a ph of 5 in particular, the amount of RNA recovered was much higher than in the case of washing with PBS having a ph of 7, and by washing the resulting solid component with an acidic buffer solution after stabilizing with nucleic acid stabilizer, a favorable nucleic acid-containing sample having extremely high nucleic acid recovery efficiency was clearly determined to be able to be prepared.

TABLE 2

| Washing Step | Stabilized | Non-Stabilized |
|---|---|---|
| (1) Washing with citric acid buffer (pH 5) | 83 | 25 |
| (2) Washing with PBS (pH 7) | 34 | 3 |
| (3) Washing step not carried out | 12 | 39 |

Amt. of RNA Recovered (μg)

In addition, the degree of degradation of the recovered RNA was investigated by electrophoresing with a bioanalyzer (Agilent Technologies). The resulting stained images are shown in FIG. 2. In the figure, “Ladder” indicates the lane in which markers were electrophoresed. Moreover, relative values of the staining intensities of the 16S rRNA and 23S rRNA bands in the stained images are shown in Table 3. Furthermore, each band intensity was calculated as the relative value based on a value of 1 for the peak area of 16S rRNA of the RNA recovered from the solid component having the largest amount of recovered RNA (solid component washed with citric acid buffer following stabilization treatment). In addition, in Table 3, the numbers (1), (2) and (3) for the washing step have, the same meanings as in Table 2. As a result, in those samples that were not stabilized with nucleic acid stabilizer, degradation of RNA ended up being promoted as a result of carrying out the washing step, and particularly as a result of washing with PBS, and an adequate amount of nucleic acid was confirmed to be unable to be recovered even if the washing step is carried out. On the other hand, in the samples stabilized with nucleic acid stabilizer, although hardly any degradation of nucleic acid was observed in the case of washing with the citric acid buffer, nucleic acid degradation was confirmed to be promoted in the case of washing with PBS. On the other hand, in the case of not carrying out the washing step, hardly any bands were detected, thereby suggesting that extraction of nucleic acid was inhibited by contaminants in the stool.

On the basis of these results, the degree of nucleic acid degradation was determined to be low and high-quality nucleic acids were clearly demonstrated to be able to be recovered in adequate amounts by treating a biological sample with a nucleic acid stabilizer followed by washing the resulting solid component with a buffer solution, and particularly an acidic buffer solution, in the manner of the preparation method of the present invention.

TABLE 3
Band Intensity

| Washing | Stabilized | | | Non-Stabilized | | |
|---|---|---|---|---|---|---|
| Step | (1) | (2) | (3) | (1) | (2) | (3) |
| 23s rRNA | 0.38 | 0.08 | 0.00 | 0.06 | 0.00 | 0.12 |
| 16S rRNA | 1.00 | 0.13 | 0.00 | 0.18 | 0.00 | 0.32 |

### Example 2

The effect of the type of buffer solution used in the washing step on the amount of nucleic acid recovered was investigated when preparing nucleic acid-containing samples from a stool according to the preparation method of the present invention using a 70% ethanol solution as nucleic acid stabilizer.

First, 1 g aliquots of a stool sample collected from a healthy subject were respectively placed in eighteen 15 mL polypropylene tubes. After dispensing, 10 mL aliquots of a 70% ethanol solution were respectively added to each of the tubes, the stool was adequately dispersed therein, and the resulting mixtures were allowed to stand undisturbed for 24 hours at 25° C. (stabilization treatment). After allowing to stand undisturbed, each of the tubes was centrifuged followed by removal of the supernatant and recovery of the solid component. The solid components were then washed using different types of washing solutions for each tube. More specifically, 10 rat of washing solution were dispensed into each solid component and after mixing well for 1 minute, the mixtures were re-centrifuged followed by recovery of the solid components. The washing solutions used consisted of citric acid/sodium hydroxide buffer (0.1 M), lactic acid/sodium lactate buffer (0.1 M) and acetic acid/sodium acetate buffer (0.1 M), having pH values ranging from 3 to 7.

RNA was recovered from each of the resulting solid components. More specifically, after adding 3 mL of the guanidine thioisocyanate solution “Buffer RLT” provided with RNeasy (Qiagen) to each tube and mixing therein, the tubes were centrifuged at 12,000×g for 20 minutes at 1° C. The supernatant obtained by the centrifugation treatment was similarly applied to the RNeasy RNA recovery column, and RNA was recovered by carrying out a washing procedure and an RNA elution procedure on the RNA recovery column in accordance with protocol provided.

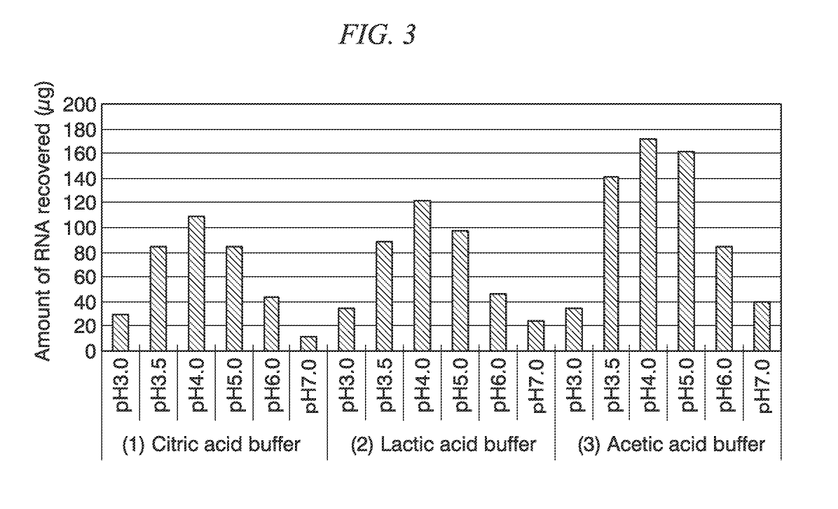

The results of quantifying the recovered RNA using Nanodrop (Nanodrop Products) are shown in Table 4 and FIG. 3. On the basis of these results, regardless of which type of buffer solution was used for the washing solution, the amount of recovered RNA was determined to be high in the vicinity of pH 4.0 to 5.0, and RNA extraction efficiency was determined to be the highest in the case of using the acetic acid/sodium acetate buffer system in particular.

TABLE 4

| | Citric Acid | Lactic Acid | Acetic Acid |
|---|---|---|---|
| | Buffer | Buffer | Buffer |
| pH 3.0 | 30 | 34 | 34 |
| pH 3.5 | 84 | 89 | 141 |
| pH 4.0 | 109 | 122 | 172 |
| pH 5.0 | 84 | 98 | 162 |
| pH 6.0 | 43 | 46 | 84 |
| pH 7.0 | 12 | 25 | 40 |

Amt. of RNA Recovered (μg)

### Example 3

Nucleic acid-containing samples were prepared from stool according to the preparation method of the present invention using a protease inhibitor as nucleic acid stabilizer.

First, 1 g aliquots of a stool sample collected from a healthy subject were respectively placed in six 15 mL polypropylene tubes. 10 mL aliquots of a 100-fold dilution of a protease inhibitor cocktail (Sigma) (solution obtained by diluting the undiluted cocktail by a factor of 100 with distilled water) were respectively added to three of the tubes, the stool was adequately dispersed therein, and the resulting mixtures were allowed to stand undisturbed for 3 hours at 25° C. (stabilization treatment). After al owing to stand undisturbed, the mixtures were centrifuged followed by removal of the supernatant and recovery of the solid component (stabilized tubes). On the other hand, the remaining three tubes were centrifuged immediately without being treated followed by removal of the supernatant and recovery of the solid component (non-stabilized tubes).

A washing step as described below was carried out on the solid components. First, 10 mL of acetic acid/sodium hydroxide buffer (0.1 M, pH 5) were dispensed into one of the three stabilized tubes, while 10 mL of PBS (phosphate buffered saline, pH 7) were dispensed into another of the tubes followed by mixing well for 1 minute, re-centrifuging and recovering the solid components. The washing step was not carried out on the remaining tube. 10 mL of acetic acid/sodium hydroxide buffer were similarly dispensed into one of the three non-stabilized tubes, 10 mL of PBS were dispensed into another of the tubes, and the washing step was not carried out on the remaining tube.

RNA was recovered from each of the resulting solid components and quantified in the same manner as Example 1. Quantification results are shown in Table 5. In Table 5, “acetic acid buffer” refers to acetic acid/sodium hydroxide buffer (0.1 M) As a result, even in the case of using the protease inhibitor as nucleic acid stabilizer, in the case of carrying out the washing step after having stabilized the nucleic acids with the protease inhibitor in the same manner as Example 1, the amount of RNA recovered was higher than in the case of not carrying out the washing step. In the case of haying washed with the pH 5 acetic acid buffer in particular, the amount of RNA recovered was much higher than in the case of washing with the pH 7 PBS, and by washing the resulting solid component with an acidic buffer solution after stabilizing with nucleic acid stabilizer, a favorable nucleic acid-containing sample having extremely high nucleic acid recovery efficiency was clearly determined to be able to be prepared.

TABLE 5

| Washing Step | Stabilized | Non-Stabilized |
|---|---|---|
| Washing with acetic acid buffer (pH 5) | 103 | 32 |
| Washing with PBS (pH 7) | 46 | 5 |
| Washing step not carried out | 15 | 42 |

Amt. of RNA Recovered (μg)

### Example 4

Nucleic acid-containing samples were prepared from stool according to the preparation method of the present invention using a saturated aqueous sodium chloride solution (saturated saltwater) as nucleic acid stabilizer. The saturated saltwater was obtained by dissolving an excess of sodium chloride in water at 50° C. followed by gradually cooling to 25° C. and using the resulting supernatant after confirming precipitation of sodium chloride.

First, 1 g aliquots of a stool sample collected from a healthy subject were respectively placed in six 15 mL polypropylene tubes. 10 mL aliquots of the saturated saltwater prepared in the manner described above were respectively added to three of the tubes, the stool was adequately dispersed therein, and the resulting mixtures were allowed to stand undisturbed for 3 hours at 25° C. (stabilization treatment). After allowing to stand undisturbed, the mixtures were centrifuged followed by removal of the supernatant and recovery of the solid component (stabilized tubes). On the other hand, the remaining three tubes were centrifuged immediately without being treated followed by removal of the supernatant and recovery of the solid component (non-stabilized tubes).

A washing step as described below was carried out on the solid components. First, 10 mL of citric acid/sodium hydroxide buffer (0.1 M, pH 5) were dispensed into one of the three stabilized tubes, while 10 mL of PBS (phosphate buffered saline, pH 7) were dispensed into another of the tubes followed by mixing well for 1 minute, re-centrifuging and recovering the solid components. The washing step was not carried out on the remaining tube. 10 mL of citric acid/sodium hydroxide buffer were similarly dispensed into one of the three non-stabilized tubes, 10 mL of PBS were dispensed into another of the tubes, and the washing step was not carried out on the remaining tube.

RNA was recovered from each of the resulting solid components and quantified in the same manner as Example Quantification results are shown in Table 6. In Table 6, “citric acid buffer” refers to citric acid/sodium hydroxide buffer (0.1 M). As a result, even in the case of using a high salinity solution such as saturated saltwater as nucleic acid stabilizer, in the case of carrying out the washing step after having stabilized the nucleic acids with the high salinity solution in the same manner as Example 1, the amount of RNA recovered was higher than in the case of not carrying out the washing step. In the case of having washed with the pH 5 citric acid buffer in particular, the amount of RNA recovered was much higher than in the case of washing with the pH 7 PBS, and by washing the resulting solid component with an acidic buffer solution after stabilizing with nucleic acid stabilizer, a favorable nucleic acid-containing sample having extremely high nucleic acid recovery efficiency was clearly determined to be able to be prepared.

TABLE 6

| Washing Step | Stabilized | Non-Stabilized |
|---|---|---|
| Washing with citric acid buffer (pH 5) | 73 | 18 |
| Washing with PBS (pH 7) | 21 | 2 |
| Washing step not carried out | 10 | 30 |

Amt. of RNA Recovered (μg)

### Example 5

After preparing nucleic acid-containing samples from stool according to the preparation method of the present invention, RNA recovered from the resulting nucleic acid-containing samples was analyzed.

More specifically, 1 g aliquots of a stool sample collected from a colon cancer patient in whom expression of Cox-2 gene, which is a marker indicating neoplasmic transformation and inflammatory digestive tract disease, had been confirmed, were first respectively placed in six 15 mL polypropylene tubes. 10 mL aliquots of an 80% ethanol solution were respectively added to three of: the tubes, the stool, was adequately dispersed therein, and the resulting mixtures were allowed to stand undisturbed for 3 hours at 25° C. (stabilization treatment), after which the mixtures were centrifuged followed by removal of the supernatant and recovery of the solid component (stabilized tubes). On the other hand, the remaining three tubes were centrifuged immediately without being treated followed by removal of the supernatant and recovery of the solid component (non-stabilized tubes). One tube of each of the three stabilized, tubes or non-stabilized tubes was washed with citric acid/sodium hydroxide buffer (0.1 M, pH 5), another tube was washed with PBS (phosphate buffered saline, pH 7), and the washing step was not carried out on the remaining tube in the same manner as Example 1. RNA was recovered from each of the resulting solid components in the same manner as Example 1.

Next, RT-PCR was carried out on the recovered RNA to detect human Cox-2 gene. The Cox-2 Primer Probe Mix manufactured by Applied Biosystems was used for the PCR primer. More specifically, 1 μL aliquots of the resulting cDNA were respectively dispensed into a 0.2 mL 96-well PCR plate. Subsequently, 8 μL of ultrapure water and 10 μL of nucleic acid amplification reagent (TaqMan Gene Expression Master Mix, Applied Biosystems) were added to each well, followed by respective addition of 1 μL aliquots of Cox-2 Primer Probe Mix (Applied Biosystems) and mixing to prepare PCR reaction solutions. The PCR plate was then placed in an ABI real-time PCR system, and PCR was carried out while measuring the fluorescence intensity over time by treating for 10 minutes at 95° C., carrying out 40 heating cycles consisting of 1 minute at 95° C., 1 minute at. 56.5° C. and 1 minute at 72° C., and finally treating for 7 minutes at 72° C. The results of analyzing the measurement results for fluorescence intensity and calculating a relative value of the expressed amount of Cox-2 in the RNA recovered from each stool sample (stabilized, based on a value of 1 for washing with the citric acid buffer) are shown in Table 7.

As shown in Table 7, the expressed amount of Cox-2 gene in the nucleic acid-containing sample washed with an acidic buffer solution following stabilization treatment was higher than the expressed amount in the nucleic acid-containing samples prepared under other conditions. Since these results correlate with the results of Example 1, use of the preparation method of the present invention was clearly determined to enable the expressed amount of Cox-2 gene in stool to be quantified efficiently due to the high nucleic acid recovery efficiency thereof.

TABLE 7

| Washing Step | Stabilized | Non-Stabilized |
|---|---|---|
| (1) Washing with citric acid buffer (pH 5) | 1.00 | 0.35 |
| (2) Washing with PBS (pH 7) | 0.38 | 0.08 |
| (3) Washing step not carried out | 0.18 | 0.49 |

Units: Relative value of Cox-2 expression level

### Reference Example 1

1 g aliquots of a stool sample collected from a healthy subject were respectively dispensed into three 15 mL polypropylene tubes. One of the tubes was rapidly frozen using liquid nitrogen immediately after dispensing to obtain stool sample (1A) 10 mL of a 70% ethanol solution were added to another tube after dispensing and the stool was dispersed well therein, followed by allowing to stand undisturbed for 1 hour at room temperature to obtain stool sample (1B). The remaining tube was promptly transferred to a washing step without adding a solvent and the like after dispensing to obtain stool sample (1C).

Subsequently, RNA was recovered from each of the stool samples. More specifically, 3 mL of a phenol mixture known as “Trizol” (Invitrogen) were added to each stool sample followed by mixing well using a homogenizer for 30 seconds or more, adding 3 mL of chloroform, again mixing well using a vortex mixer, and centrifuging at 12,000×g for 20 minutes at 4° C. The supernatant obtained by centrifugation (aqueous layer) was applied to an RNA recovery column (RNeasy Midi Kit, Qiagen), and RNA was recovered by carrying out a washing procedure and an RNA elution procedure on the RNA recovery column in accordance with the protocol provided. The recovered RNA was quantified using Nanodrop (Nanodrop Products).

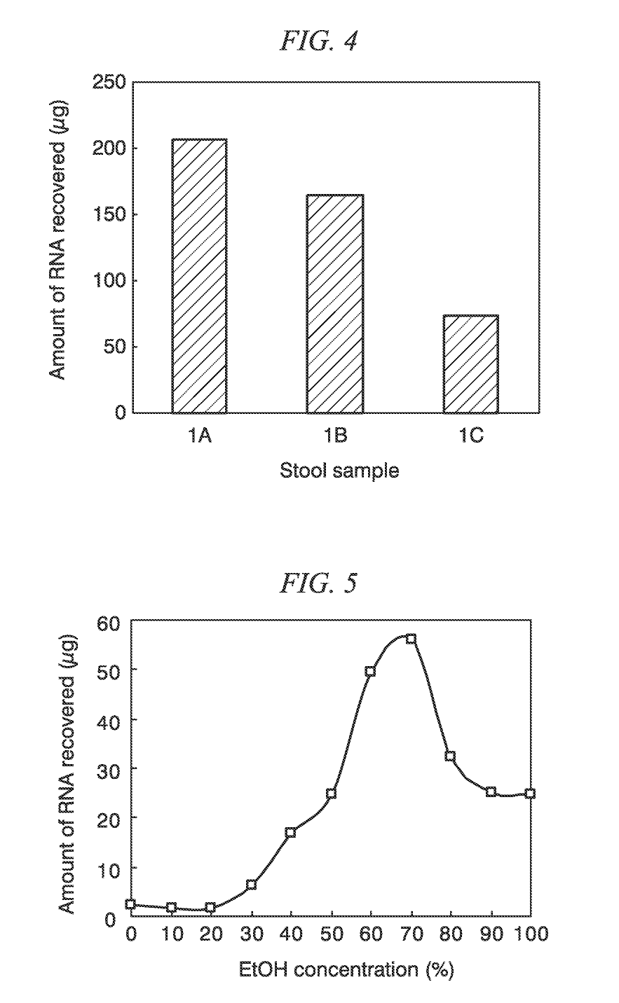

FIG. 4 shows the amounts of RNA recovered from each of the stool samples. Although the amount of RNA recovered from stool sample (1B) prepared using an ethanol solution was slightly less than the amount of RNA recovered from stool sample (1A) that was frozen immediately after collection, extremely large amounts of RNA were able to be recovered in comparison with stool sample (1C) on which nucleic acid extraction was carried out soon after collection. On the basis of these results, preparation using a water-soluble organic solvent as nucleic acid stabilizer in the present invention clearly allows the obtaining of stool samples from which nucleic acids can be recovered extremely efficiently even if prepared at room temperature. Although it desirable that stool samples be able to be prepared in the vicinity of room temperature in the case of patients collecting stool samples at home as in the case of health examinations and the like, stabilization treatment of stool samples with a water-soluble organic solvent makes it possible to effectively respond to such requirements.

### Reference Example 2

Stool samples were prepared by using a mixture containing 5.0×10⁵ Caco-2 human colon cancer-derived cultured cells, which highly express multi-drug resistance 1 (MDR1) gene, in 0.5 g of stool collected from a healthy subject for use as pseudo colon cancer patient stool samples.

More specifically, 0.5 aliquots of the pseudo colon cancer patient stool were dispensed into 15 ml polyproylene tubes followed by the respective addition of the stool sample preparation solutions listed in Table 8 and mixing to prepare stool samples. Furthermore, in the table, “universal collection medium” refers to the storage medium described in Patent Document 4 (500 mL of Pack Saline G, 400 mg of sodium bicarbonate, 10 g of BSA, 500 units/L of penicillin G, 500 mg/L, of streptomycin sulfate, 1.25 mg/L of amphotericin B, and 50 mg/L of gentamicin). The prepared stool samples were stored for 1, 3, 7 or 10 days, respectively, in a constant temperature incubator at room temperature (25° C.).

TABLE 8
Stool Sample Preparation Solution

| | |
|---|---|
| (2A) | 5 ml of 70% ethanol solution |
| (2B) | 1 mL of 100% methanol solution |
| (2C) | 5 mL of universal collection medium |
| (2D) | 5 mL of PBS |

Following storage, RNA was recovered from each of the stool samples, and mRNA, which is the transcription product of the MDR1 gene, was attempted to be detected for the recovered RNA. In the case of the stool sample prepared using stool sample preparation solution (2C) (referred to as stool sample (2C)), RNA was recovered after first isolating mammalian cells including the Caco-2 cells. In the case of the stool samples prepared using stool sample preparation solutions other than stool sample preparation solution (2C), mammalian cell-derived nucleic acids and bacteria-derived nucleic acids were recovered simultaneously without isolating mammalian cells. Isolation of mammalian cells from the stool sample (2C) specifically consisted of adding 5 mL of Histopaque 1077 solution (Sigma) to the stool sample (2C) and mixing followed by centrifuging at 200×g for 30 minutes at room temperature and recovering the interface between the culture liquid and the Histopaque 1077 solution. The isolated mammalian cells were washed three times with PBS.

Recovery of RNA from the stool samples was specifically carried out in the manner described below. First, 3 mL of a phenol mixture “Trizol” (Invitrogen) were added to the stool samples (only to isolated mammalian cells in the case of stool sample (2C)), and after mixing well with a homogenizer for 30 seconds or more, 3 mL of chloroform were added followed by centrifuging at 12,000×g for 10 minutes. The supernatant (aqueous layer) obtained, by the centrifugation was recovered into fresh polypropylene tubes. Subsequently, RNA was recovered from the recovered supernatant using the RNeasy Kit (Qiagen).

RT-PCR was carried out on the recovered RNA, and PCR was carried out using the resulting cDNA as template. The primers used consisted of a forward primer for MDR1 gene amplification having the base sequence of SEQ ID NO. 1 and a reverse primer for MDR1 acne amplification having the base sequence of SEQ ID NO. 2.

More specifically, 12 μL of ultrapure water and 2 μL of 10× buffer were added to 0.2 mL PCR tubes followed by the further addition of 1 μL aliquots each of the cDNA, forward primer, reverse primer, magnesium chloride, dNTP and DNA polymerase and mixing to prepare PCR reaction solutions. PCR was then carried out by subjecting the PCR tubes to reaction conditions consisting of 30 cycles of 30 seconds at 95° C., 30 seconds at 60° C. and 1 minute at 72° C. As a result, the resulting PCR products were phoresed using the Agilent DNA1000 LabChip® Kit (Agilent Technologies) followed by measuring the intensities of the resulting bands to investigate the degree of amplification of the PCR products.

TABLE 9

Storage Period

| | 1 day | 3 days | 7 days | 10 days |
|---|---|---|---|---|
| Stool sample (2A) | ++ | ++ | ++ | + |
| Stool sample (2B) | ++ | ++ | + | + |
| Stool sample (2C) | − | − | − | − |
| Stool sample (2D) | + | − | − | − |

++: Strong amplification,
+: Moderate amplification,
+/−: Weak amplification,
−: No amplification

Table 9 summarizes the degrees of amplification of PCR products derived from each of the stool samples for each storage period. Furthermore, in the table, “stool sample (2A)” refers to the stool sample prepared using the stool sample preparation (2A), “stool sample (2B)” refers to the stool sample prepared using the stool sample preparation (2B), and “stool sample (2D)” refers to the stool sample prepared using the stool sample preparation solution (2D).

As a result, in the case or stool sample (2D), although amplification of the PCR product was confirmed in the case of a storage period of 1 day, amplification was unable to be confirmed starting at a storage period of 3 days. In contrast, in the case of the stool sample (2A) and the stool sample (2B), which were prepared using the stool sample preparation solution (2A) and the stool sample preparation solution (2B) that are stool sample preparation solutions of the present invention, amplification of the PCP product was able to be confirmed even after a storage period of 10 days. On the other hand, in the case of the stool sample (2C), which was prepared using the stool sample preparation solution (2C) described in Patent Document 4, amplification of the PCR product was unable to be confirmed even after a storage period of 1 day.

On the basis of the above results, treatment of stool using a water-soluble organic solvent as nucleic acid stabilizer as in the present invention enables nucleic acids contained in stool to be recovered efficiently, and as a result thereof, the accuracy of RNA analysis was clearly determined to be able to be improved. This is presumed to be because the use of a water-soluble organic solvent makes it possible to stabilize and preserve nucleic acids derived from mammalian cells contained in stool, and even RNA that is particularly susceptible to degradation, so as to be able to be stored for a long period of time at room temperature.

On the other hand, since amplification of the POP product derived from stool sample (2C) was not confirmed, in the case of treating stool using a solution containing an antibiotic instead of a nucleic acid stabilizer, although bacterial cells present in stool are eliminated by the antibiotic, the possibility is suggested that RNA degradation may be promoted due to the release of RNase and the like from the killed bacterial cells. In addition, due to the small number of mammalian cells contained in stool, in the case of isolating mammalian cells from stool, the possibility is suggested that it may be difficult to recover an adequate amount of nucleic acid in comparison with the nucleic acid recovery method of the present invention in which nucleic acids derived from bacterial cells are able to function as carriers.

### Reference Example 3

0%, 10%, 20%, 30%, 40%, 50%, 60%, 70%, 80%, 90% and 100% ethanol solutions were respectively prepared by diluting with ultrapure water. 5 mL aliquots of these ethanol solutions were respectively dispensed into 15 mL polypropylene tubes.

0.5 aliquots of stool collected from a healthy subject were respectively placed in each of the tubes followed by allowing to stand undisturbed for 48 hours at 37° C. Subsequently, each of the tubes was centrifuged followed by removing the supernatant, adding 3 mL of a phenol mixture “Trizol” (Invitrogen) to the resulting solid component, mixing well with a homogenizer for 30 seconds or more, adding 3 mL of chloroform, and centrifuging at. 12,000×g for 10 minutes. The supernatant. (aqueous layer) obtained by the centrifugation was collected into fresh polypropylene tubes. Subsequently, RNA was recovered from the recovered supernatant using the RNeasy Midi Kit (Qiagen).

FIG. 5 indicates the recovered amounts of RNA from the stool samples prepared using the various concentrations of ethanol solutions. As a result, in the case of using an alcohol such as ethanol as nucleic acid stabilizer, the alcohol concentration was clearly determined to preferably be 30% or more, more preferably 50% or more, even more preferably 50% to 80%, and particularly preferably 60% to 70%.

### Reference Example 4

Stool collected from five healthy subjects was mixed and 0.2 g aliquots thereof were respectively placed in two 15 mL polypropylene tubes 10 mL of a 32% denatured alcohol solution containing 18% isopropanol (50% in terms of the total alcohol solution) were added to one of the tubes followed by mixing well and allowing to stand undisturbed for 1 day at 25° C. This stool sample was designated as stool sample (A4). The remaining tube was used as a control, and was stored in a deep freezer at −80° C. immediately after dispensing.

DNA was recovered from both stool samples using the DNA extraction kit ° QIAmp DNA Stool Mini Kit (Qiagen). As a result of quantifying the concentration of the recovered DNA by absorptiometry, nearly equal amounts of DNA were able to be recovered from both stool samples.

A mutation analysis was carried out on 100 ng of the recovered DNA using the K-ras gene mutation analysis kit. “K-ras Codon 12 Mutation on Detection Reagent.” (Wakunaga Pharmaceutical) in accordance with the protocol provided. As a result, the DNA recovered from the stool sample (A4) was determined to be negative for all six types of mutant genes in the same manner as in the case of using DNA recovered from the control sample.

on the basis of the above results, the use of nucleic acid recovered from a nucleic acid-containing sample obtained by treating collected stool with a nucleic acid stabilizer such as a water-soluble organic solvent clearly enabled nucleic acid analysis to be carried out accurately even if the analysis requires a high degree of accuracy with respect to gene mutation and the like. In addition, although denatured ethanol consisting of a mixture of isopropanol and ethanol was used for the nucleic acid stabilizer in this example, similar results were obtained using a 50% ethanol solution having the same alcohol concentration.

### Reference Example 5

0.1 g aliquots of stool collected from a healthy subject were respectively placed in three 15 mL polypropylene tubes, 3 mL of 70% ethanol were added to one of the tubes followed by adequately dispersing the stool therein, and the resulting stool sample was designated as stool sample (5A). On the other hand, 2.4 mL each of “Isogen” (Nippon Gene) were added to the remaining two tubes followed by dispersing the stool therein, and the resulting stool samples were designated as comparative sample (P1) and comparative sample (P2), respectively. Furthermore, “Isogen” is a phenol-containing reagent containing 40% phenol (solubility in water approx. 10% by weight).

RNA was recovered from comparative sample (P1) immediately after dispersing the stool. More specifically, after adequately mixing the stool sample with a homogenizer for 30 seconds or more, 3 mL of chloroform were added followed by centrifuging at 12,000×g for 10 minutes. The supernatant (aqueous layer) obtained by the centrifugation was recovered in a fresh polypropylene tube. Subsequently, RNA was recovered from the recovered supernatant using the RNeasy Midi Kit (Qiagen).

In addition, comparative sample (P2) was allowed to stand undisturbed for 5 hours at room temperature followed by recovering RNA in the same manner as comparative sample (P1).

On the other hand, after allowing the stool sample (5A) to stand undisturbed for 5 hours at room temperature in the same manner as comparative sample (P2), it was centrifuged followed by removing the supernatant, adding 2.4 mL of “Isogen” to the resulting precipitate (solid component) and recovering RNA in the same manner as comparative sample (P1).

The recovered RNA was quantified using Nanodrop (Nanodrop Products). As a result, although 32 μg of RNA were able to be recovered from the comparative sample (P1) from which RNA was recovered immediately after stool sample preparation, only 14 μg were able to be recovered from the comparative sample (P2) on which the recovery procedure was carried out after allowing to stand undisturbed for 5 hours at room temperature. In contrast, 57 μg of RNA, which is larger than the amount recovered from the comparative sample (P1), were able to be recovered from the stool sample (5A) even though the recovery procedure was carried out after allowing to stand for 5 hours at room temperature.

On the basis of these results, the use of a nucleic acid stabilizer clearly made it possible to recover RNA extremely efficiently in comparison with the case of using a conventional phenol solution.

### INDUSTRIAL APPLICABILITY

According to the method of preparing samples containing nucleic acid of the present invention, since a nucleic acid-containing sample, from which nucleic acids in a biological sample can be efficiently recovered, can be prepared easily, the method of the present invention can be used in fields such as periodic health examinations and other forms of clinical testing that involve the use of biological samples in particular.

1. A method of preparing a nucleic acid-containing sample from a biological sample, comprising:
(A) mixing a biological sample with a nucleic acid stabilizer to obtain a mixture,
(B) recovering a solid component from the mixture obtained in (A) to obtain a nucleic acid-containing sample, and
(C) washing the solid component recovered in (B) using an acidic buffer solution having a pH of 2 to 14.

2. The method of preparing a nucleic acid-containing sample according to claim 1, wherein the pH of the acidic buffer solution is 3 to 6.

3. The method of preparing a nucleic acid-containing sample according to claim 1, wherein the nucleic acid stabilizer is at least one of a water-soluble organic solvent, a protease inhibitor, a polycation, and a hypertonic solution.

4. The method of preparing a nucleic acid-containing sample according to claim 3, wherein the water-soluble organic solvent contains at least one of a water-soluble alcohol, ketone, and an aldehyde.

5. The method of preparing a nucleic acid-containing sample according to claim 4, wherein the water-soluble alcohol is ethanol, propanol or methanol.

6. The method of preparing a nucleic acid-containing sample according to claim 4, wherein the ketone is acetone or methyl ethyl ketone.

7. The method of preparing a nucleic acid-containing sample according to claim 3,
wherein the nucleic acid stabilizer is a water-soluble organic solvent, and
wherein the concentration of the water-soluble organic solvent in the mixture is 30% or more.

8. The method of preparing a nucleic acid-containing sample according to claim 3,
wherein the nucleic acid stabilizer is a water-soluble organic solvent, and
wherein the concentration of the water-soluble organic solvent in the mixture is 0.01% to 30%.

9. The method of preparing a nucleic acid-containing sample according to claim 3, wherein the protease inhibitor is at least one of a peptide-based protease inhibitor, a reducing agent, a protein denaturing agent, and a chelating agent.

10. The method of preparing a nucleic acid-containing sample according to claim 3, wherein the protease inhibitor is AEBSF, aprotinin, bestatin, E-64, leupeptin, pepstatin A, urea, dithiothreitol (DTT) or EDTA.

11. The method of preparing a nucleic acid-containing sample according to claim 3, wherein the polycation is polylysine.

12. The method of preparing a nucleic acid-containing sample according to claim 1, wherein the acidic buffer solution is a buffer solution selected from the group consisting of an acetic acid/sodium acetate buffer system, a citric acid/sodium hydroxide buffer system and a lactic acid/sodium lactate buffer system.

13. The method of preparing a nucleic acid-containing sample according to claim 1, wherein the pH of the acidic buffer solution is 3.5 to 5.5.

14. The method of preparing a nucleic acid-containing sample according to claim 13, wherein the pH of the acidic buffer solution is 4.0 to 5.0.

15. The method of preparing a nucleic acid-containing sample according to claim 1, wherein the mixture in (A) further comprises a surfactant.

16. The method of preparing a nucleic acid-containing sample according to claim 1, wherein the mixture in (A) further comprises a colorant.

17. The method of preparing a nucleic acid-containing sample according to claim 1, wherein the biological sample is stool, blood or urine.

18. A nucleic acid-containing sample prepared according to the method of preparing a nucleic acid-containing sample according to claim 1.

19. A method of recovering a nucleic acid from a nucleic acid-containing sample prepared from a biological sample using the method of preparing a nucleic acid-containing sample claim 1, comprising:
simultaneously recovering nucleic acids derived from all biological species contained in the biological sample.

20. A method of recovering a nucleic acid from a nucleic acid-containing sample prepared from stool using the method of preparing a nucleic acid-containing sample claim 1, comprising:
simultaneously recovering a nucleic acid derived from normal intestinal bacterial flora and nucleic acid derived from an organism other than normal intestinal bacterial flora.

21. The method of recovering a nucleic acid according to claim 20, wherein the organism other than normal intestinal bacterial flora is a mammalian cell.

22. The method of recovering a nucleic acid according to claim 19, wherein the simultaneous recovering of nucleic acids comprises:
(a) denaturing protein present in the nucleic acid-containing sample and eluting nucleic acids from cells derived from all biological species contained in the nucleic acid-containing sample, and
(b) recovering nucleic acids eluted in (a).

23. The method of recovering a nucleic acid according to claim 22, wherein the simultaneous recovering of nucleic acids further comprises:
(c) removing the protein denatured in (a),
Wherein (c) is carried out after (a) and before (b).

24. The method of recovering a nucleic acid according to claim 22, wherein the denaturing of protein in (a) is carried out using one or more types of denaturing agents selected from the group consisting of a chaotropic salt, an organic solvent and a surfactant.

25. The method of recovering a nucleic acid according to claim 24, wherein the organic solvent is phenol.

26. The method of recovering nucleic acids according to claim 23, wherein the removing of the protein in (c) is carried out using chloroform.

27. The method of recovering a nucleic acid according to claim 22, wherein the recovering of nucleic acid in (b) comprises
(b1) adsorbing the nucleic acid eluted in (a) to an inorganic support, and
(b2) eluting the nucleic acid adsorbed in (b1) from the inorganic support.

28. The method of recovering a nucleic acid according to claim 22, further comprising:
(d) recovering a solid component from the nucleic acid-containing sample before (a).

29. A method of analyzing a nucleic acid, comprising:
analyzing a nucleic acid derived from a mammalian cell by using the nucleic acid recovered from a nucleic acid-containing sample using the method of recovering a nucleic acid according to claim 20.

30. The method of analyzing a nucleic acid according to claim 29, wherein the mammalian cell is a digestive tract cell.

31. The method of analyzing a nucleic acid according to claim 29, wherein the mammalian cell is an exfoliated large intestine cell.

32. The method of analyzing a nucleic acid according to claim 29, wherein the nucleic acid derived from the mammalian cell is a marker indicating a neoplasmic transformation.

33. The method of analyzing a nucleic acid according to claim 29, wherein the nucleic acid derived from the mammalian cell is a marker indicating an inflammatory digestive tract disease.

34. The method of analyzing a nucleic acid according to claim 29, wherein the nucleic acid derived from the mammalian cell is a nucleic acid derived from COX-2 gene.

35. The method of analyzing a nucleic acid according to claim 29, wherein the analysis is one or more types selected from the group consisting of mRNA expression analysis, K-ras gene mutation analysis and DNA methylation analysis.
